(19) United States
(12) Patent Application Publication
(10) Pub. No.: US 2023/0233514 A1
(43) Pub. Date: Jul. 27, 2023

(54) ANTIVIRAL COMPOUNDS AND METHODS OF USING THE SAME

(71) Applicant: FLAGSHIP PIONEERING, INC., Cambridge, MA (US)

(72) Inventors: Hok Hei Tam, Newton, MA (US); David Barry Kolesky, Arlington, MA (US)

(21) Appl. No.: 18/011,832

(22) PCT Filed: Jun. 23, 2021
(86) PCT No.: PCT/US2021/038600

§ 371 Date: Dec. 20, 2022

Related U.S. Application Data
(60) Provisional application No. 63/043,024, filed on Jun. 23, 2020.
(60) Provisional application No. 63/043,048, filed on Jun. 23, 2020.
(60) Provisional application No. 63/043,054, filed on Jun. 23, 2020.
(60) Provisional application No. 63/043,059, filed on Jun. 23, 2020.
(60) Provisional application No. 63/043,065, filed on Jun. 23, 2020.

(51) Int. Cl. A61K 31/352; A61P 31/14; A61K 31/48; A61K 31/122; A61K 31/7048; A61K 31/366; A61K 31/675

(52) U.S. Cl. CPC A61K 31/352; A61P 31/14; A61K 31/48; A61K 31/122; A61K 31/7048; A61K 31/366; A61K 31/675

(57) ABSTRACT

Anti-viral compounds and methods of using antiviral compounds are described. The compounds can be used in methods of reducing infection rate of a virus and in methods of treating a viral infection in a subject in need thereof. The virus can be a coronavirus, such as SARS-CoV-2.

### RELATED APPLICATIONS

This application claims the benefit of U.S. Provisional Application No. 63/043,024, filed on Jun. 23, 2020; U.S. Provisional Application No. 63/043,048, filed on Jun. 23, 2020; U.S. Provisional Application No. 63/043,054, filed on Jun. 23, 2020; U.S. Provisional Application No. 63/043,059, filed on Jun. 23, 2020; and U.S. Provisional Application No. 63/043,065, filed on Jun. 23, 2020. The entire teachings of the above applications are incorporated herein by reference.

### BACKGROUND

Coronaviruses are a large family of viruses that are common in people and many different species of animals. Some coronaviruses, such as SARS-CoV-2, can cause severe illness, particularly respiratory illness, in people.

### SUMMARY

The present invention relates to antiviral compounds and pharmaceutical compositions comprising such compounds; methods of reducing viral infection, replication, or virulency with the compounds and compositions, e.g., in a subject in need thereof; and methods of treating a viral infection with the compounds and compositions in a subject in need thereof. The compounds described herein may inhibit viral proteases and are useful as antiviral agents, e.g., against a coronavirus infection, e.g., infection with SARS coronavirus (SARS-CoV), which causes severe acute respiratory syndrome (SARS); MERS coronavirus (MERS-CoV), which causes Middle East respiratory syndrome (MERS); and SARS-CoV-2, which causes coronavirus disease 2019 (COVID-19) (also referred to as “2019 novel coronavirus” or “2019-nCoV”).

In one aspect, the invention provides a pharmaceutical composition comprising a compound selected from the compounds of Table 1 or Table 3, or a pharmaceutically acceptable salt thereof, and a pharmaceutically suitable carrier.

In another aspect the invention provides a method of reducing viral infection, replication, and/or virulency in a subject, comprising administering to the subject a pharmaceutical composition comprising a compound selected from the compounds of Table 1 or Table 3, or a pharmaceutically acceptable salt thereof, and a pharmaceutically suitable carrier.

In another aspect the invention provides a method of treating a subject who is diagnosed with a coronavirus-related disease, e.g., SARS, MERS, or COVID-19, the method comprising administering to the subject a pharmaceutical composition comprising a compound selected from the compounds of Table 1 or Table 3, or a pharmaceutically acceptable salt thereof, and a pharmaceutically suitable carrier.

The compounds described herein are suitable for monotherapy or suitable for use in combination therapy. For example, the methods described herein may include administering a plurality of the compounds described herein, e.g., a plurality of compounds listed in Table 1 or Table 3. In some embodiments a compound described herein is administered to a subject in combination with a second agent, e.g., a second anti-viral compound, an anti-inflammatory agent, an anticoagulant, or an analgesic. In some embodiments a plurality of compounds described herein, e.g., a plurality of compounds listed in Table 1 or Table 3, are administered to a subject in combination with another agent, e.g., another anti-viral compound, an anti-inflammatory agent, an anticoagulant, or an analgesic.

In embodiments, the other or second agent is e.g., Favilavir, Galidesivir, Remdesivir, Ifenprodil, Lopinavir and ritonavir, BPI-002; a steroid, e.g., dexamethasone; an anti-coagulant, e.g., heparin or enoxaparin; an antibody, e.g., an antibody against human granulocyte-macrophage colony-stimulating factor (GM-CSF), e.g., TJM2, Gimsilumab; an antibody against human interleukin-6 (IL-6) or IL-6R, e.g., tocilizumab or AT-100 (rhSP-D); an antibody against CCR5, e.g., leronlimab, or combinations thereof. In embodiments, the other or second agent is convalescent plasma from a human who has been infected with a coronavirus described herein. In embodiments, the second agent is selected from the compounds of Table 2, or a pharmaceutically acceptable salt thereof.

The compounds described herein (e.g., compounds of Table 1 or Table 3) can be administered separately from the second agent (e.g., compounds of Table 2). The compound of Table 1 or Table 3 can be formulated in a separate pharmaceutical composition from the compound of Table 2. When formulated separately, the pharmaceutical compositions can be concomitantly or sequentially administered by any means that achieve their intended purpose.

In other embodiments, a single pharmaceutical composition can include one or more compound described here (e.g., Table 1 or Table 3) and also one or more of the second agents (e.g., Table 2).

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-B are cell viability curves of Vero E6 cells treated with various compounds (0.001-1000 μM or vehicle control (DMSO)) for 48 hours, as assessed by CellTiterGlo. Data is normalized to untreated controls.

FIGS. 2A-O show the effect of pre-treatment with various concentrations of food-derived compounds on relative levels of SARS-CoV-2 viral load in Vero E6 cells and on host cell number. Viral load is indicated by relative levels of SARS-CoV-2 N protein immunofluorescent staining. Host Cell Number is indicated by relative levels of DAPI-positive immunofluorescent staining.

FIGS. 3A-G are dose response curve for several food-derived compounds against SARS-CoV-2 main protease (Mpro).

FIGS. 4A-G are dose response curve for food-derived compounds against SARS-CoV-2 papain-like protease (PLpro).

FIGS. 5A-F are graphs showing the effect of food-derived compounds in combination with Remdesivir on relative host cell viability of SARS-CoV-2-infected Vero E6 cells.

### DEFINITIONS

As used herein, a “pharmaceutical composition” or “pharmaceutical preparation” is a composition or preparation having pharmacological activity or other direct effect in the mitigation, treatment, or prevention of disease, and/or a finished dosage form or formulation thereof and which is indicated for human use.

As used herein, the term “pharmaceutically acceptable carrier or excipient” means a formulation auxiliary that is suitable for use in humans and is generally non-toxic or inert. A pharmaceutically acceptable carrier or excipient may be liquid, solid, or semi-solid filler, diluent, or encapsulating material. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such as propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

As used herein, the term “pharmaceutically acceptable salt” refers to those salts which are within sound medical judgment, suitable for use in contact with the tissues of humans and other mammals, and are commensurate with a reasonable benefit/risk ratio. Pharmaceutically acceptable salts are well known in the art and are described, e.g., in Gupta et al., 2018, Salts of Therapeutic Agents: Chemical, Physicochemical, and Biological Considerations. Molecules 23:1719.

As used herein, the term “subject” means an animal, preferably a mammal, e.g., a human, or a veterinary or agricultural animal. In embodiments, the subject is a non-human mammal such as a non-human primate (e.g., monkeys, apes), ungulate (e.g., cattle, buffalo, sheep, goat, pig, camel, llama, alpaca, deer, horses, donkeys), carnivore (e.g., dog, cat), rodent (e.g., rat, mouse), or lagomorph (e.g., rabbit).

As used herein, the terms “effective amount,” “therapeutically effective amount,” and a “sufficient amount” of a composition described herein refer to a quantity sufficient to, when administered to a subject, including a mammal (e.g., a human), effect beneficial or desired results, including effects at the cellular level, tissue level, or clinical results, and, as such, an “effective amount” or synonym thereto depends upon the context in which it is being applied. The amount of a given composition described herein that will correspond to such an amount will vary depending upon various factors, such as the given agent, the pharmaceutical formulation, the route of administration, the type of disease or disorder, the identity of the subject (e.g., age, sex, weight) or host being treated, and the like, but can nevertheless be routinely determined by one skilled in the art.

As used herein, “treatment” and “treating” refer to the medical management of a subject with the intent to improve, ameliorate, stabilize (i.e., not worsen), prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to minimizing or partially or completely inhibiting the development of the associated disease, pathological condition, or disorder); and supportive treatment (treatment employed to supplement another therapy). Treatment also includes diminishment of the extent of the disease or condition (e.g., reducing viral infection, replication, and/or virulency); preventing spread of the disease or condition; delay or slowing the progress of the disease or condition; amelioration or palliation of the disease or condition; and remission (whether partial or total), whether detectable or undetectable. “Ameliorating” or “palliating” a disease or condition means that the extent and/or undesirable clinical manifestations of the disease, disorder, or condition are lessened and/or time course of the progression is slowed or lengthened, as compared to the extent or time course in the absence of treatment. “Treatment” can also mean prolonging survival as compared to expected survival if not receiving treatment. Those in need of treatment include those already with the condition or disorder, as well as those at risk to have the condition or disorder or those in which the condition or disorder is to be prevented.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In other embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

### DETAILED DESCRIPTION

A description of example embodiments follows.

### Overview of Methodology

A large scale in silico screen involving artificial intelligence (AI) algorithms was performed to identify compounds that may inhibit viral proteases. Approximately 40,000 compounds were screened for the ability to decrease viral replication of the novel SARS-CoV-2 coronavirus, and the compounds described herein were selected for further investigation.

Of those compounds selected for further investigation, only a few exhibited anti-viral activity. Further testing demonstrated that these compounds inhibit the activity of two isolated SARS CoV-2 proteases, the main protease (Mpro) and papain-like protease (PLpro), in a direct enzyme assay. The main protease (Mpro) is required to cleave viral polyproteins, including those required for viral replication. Inhibition of this protease prevents the virus from replicating marking it as a possible therapeutic target for preventing or treating SARS-CoV-2 infection (Sacco et al. (2020); Coelho et al. (2020)). Similarly, the papain-like protease (PLpro) is required for polyprotein processing and represents an alternative therapeutic target for the treatment of SARS CoV-2 infections (Klemm et al. (2020)).

The in silico screening and laboratory experiments highlight the challenges in identifying compounds that are effective in inhibiting viral replication. Approximately 40,000 compounds were screened in silico in order to identify compounds for further investigation. Yet of those tested, only a few reduced viral load and inhibited the viral proteases, Mpro and/or PLpro.

### Coronaviruses

Coronaviruses are a large family of viruses that are common in people and many different species of animals. Some coronaviruses can cause severe illness in people. Some notable coronaviruses include SARS coronavirus (SARS-CoV), which causes severe acute respiratory syndrome (SARS); MERS coronavirus (MERS-CoV), which causes Middle East respiratory syndrome (MERS); and SARS-CoV-2, which causes coronavirus disease 2019 (COVID-19).

Infection with a coronavirus can cause fever, cough, and shortness of breath. Infection can be particularly dangerous in older people, people with weakened immune systems, and people with underlying health conditions, such as cardiovascular disease, diabetes, and chronic lung disease, among others.

### Antiviral Compounds

Based on an in silico screen, the compounds of Table 1 were identified as compounds that may be effective in inhibiting a viral protease of a coronavirus. In one embodiment of any aspect of the invention, the compound is a compound listed in Table 1 or a pharmaceutically acceptable salt thereof.

TABLE 1
Antiviral Compounds

| Name | Structure |
|---|---|
| Hypericin | 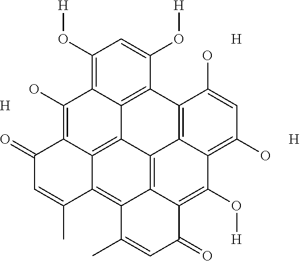 |
| Fagopyrine | 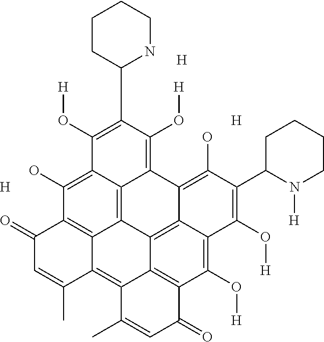 |
| Protohypericin | 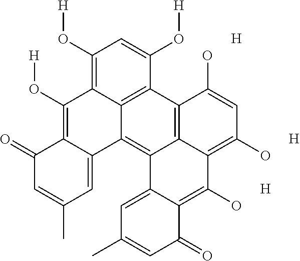 |
| Pseudohypericin | 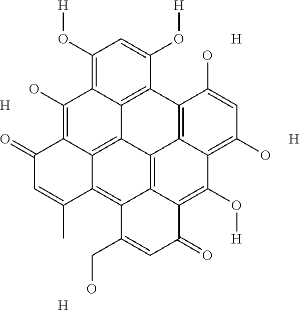 |
| Artonin A | 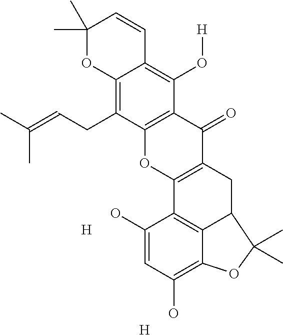 |
| Trisjuglone | 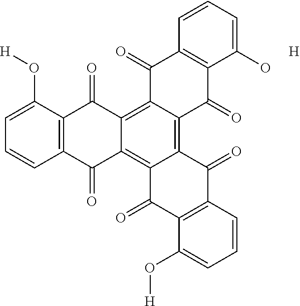 |
| Casuarictin | 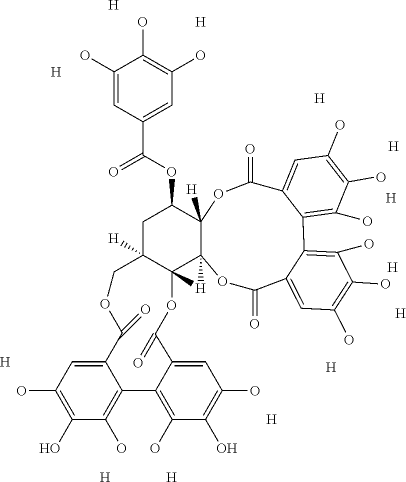 |
| Tellimagrandin 1 | 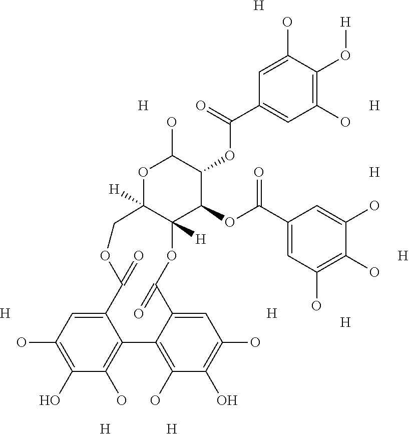 |
| Alnusiin | 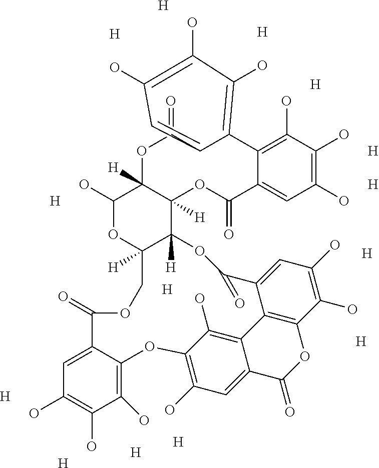 |
| Vescalagin | 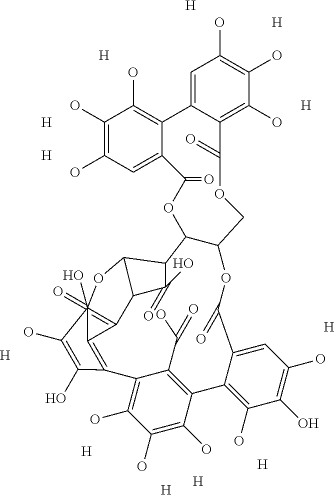 |
| Punicalagin | 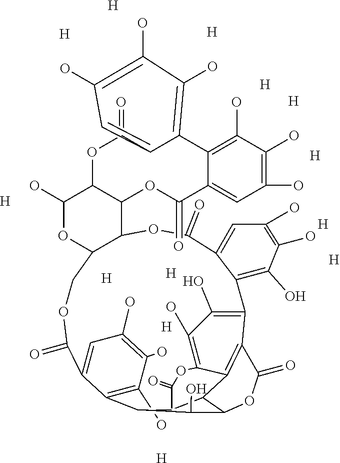 |
| Theaflavin | 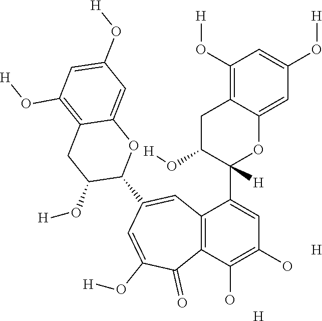 |
| Theaflavin 3-gallate | 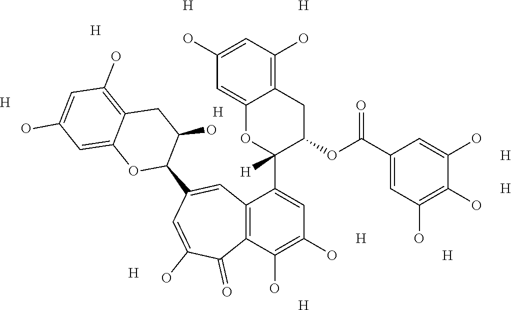 |
| Hinokiflavone | 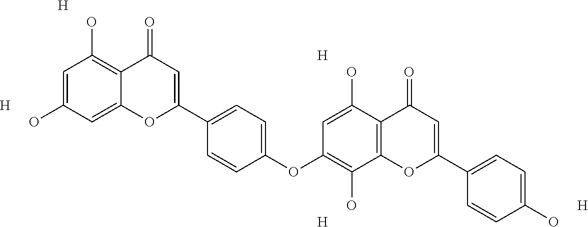 |
| Ginkgetin | 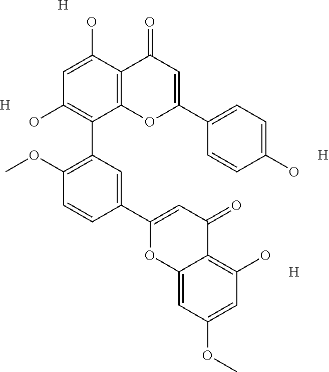 |
| Podocarpusflavone A | 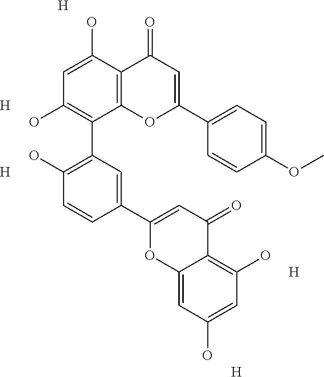 |
| Sequoiaflavone | 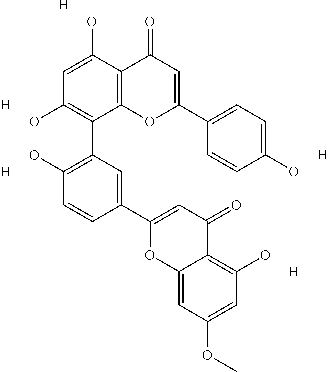 |
| Sotetsuflavone | 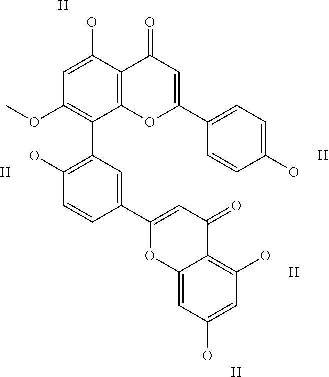 |
| Taiwanhomoflavone A | 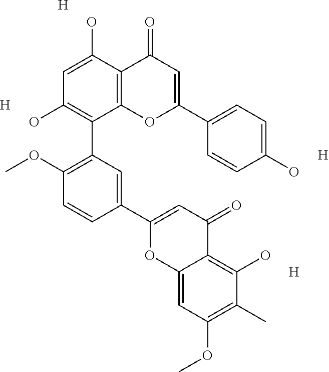 |
| Amentoflavone | 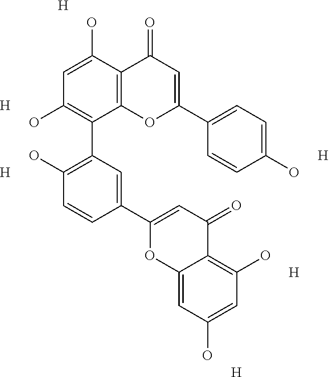 |
| Bilobetin | 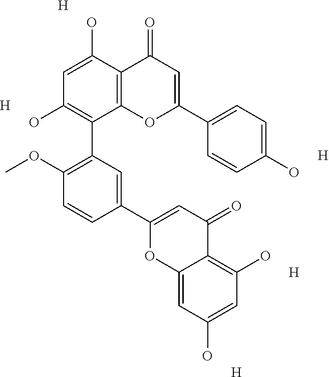 |
| Smitilbin | 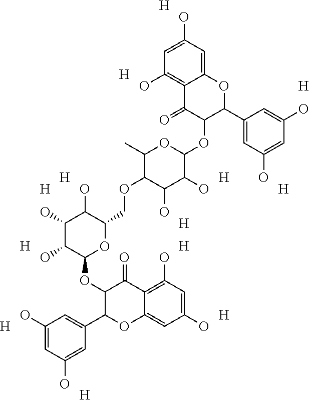 |
| Diathin F | 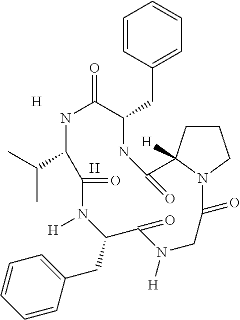 |
| 2-Phloreckol | 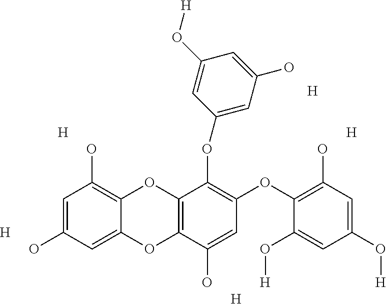 |
| Ergotamine | 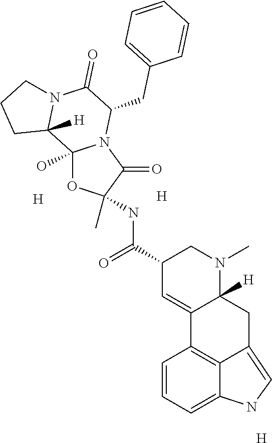 |
| Bismahanine | 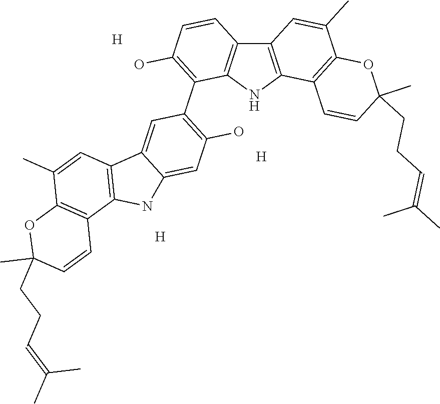 |
| Lactucain C | 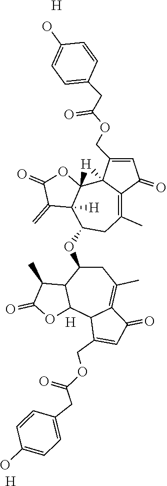 |
| Jugnaphthalenoside C | 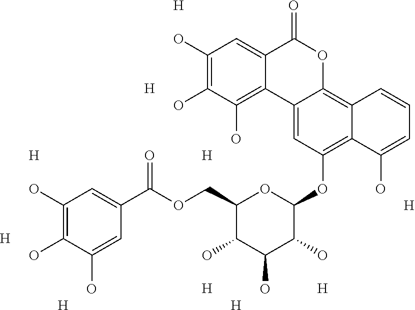 |
| Usambarensine | 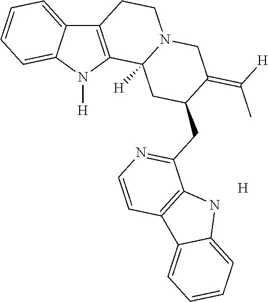 |
| Grandione | 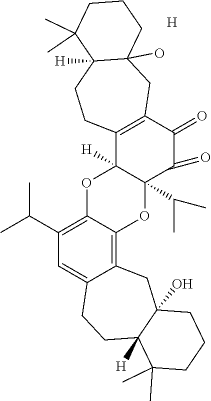 |
| Neoacrimarine H | 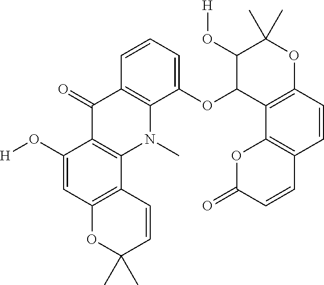 [SMILES: Cn1c2c(OC3c4c(ccc5ccc(=O)oc45)OC(C)(C)C3O)cccc2c(=O)c2c(O)cc3c(c21)C=CC(C)(C)O3] |
| Ormosinine | 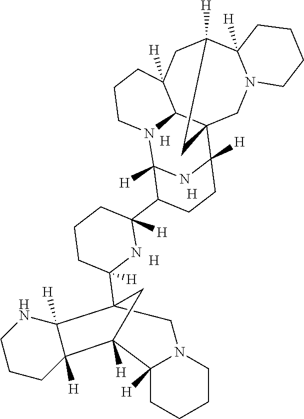 |
| Mulberrofuran M | 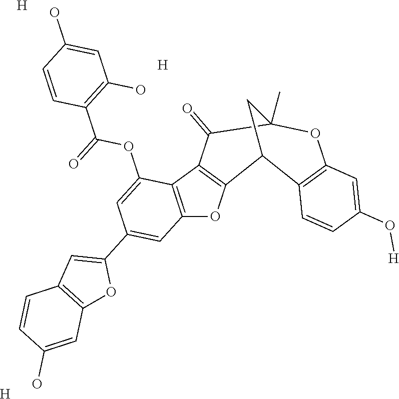 |
| Castacrenin A | 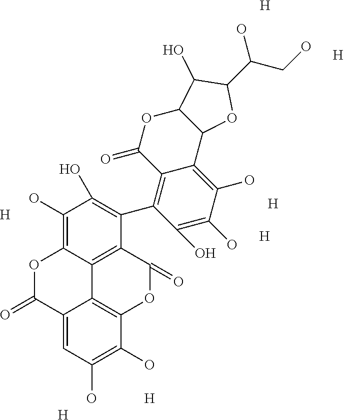 |
| Ridiculuflavonyl- chalcone A | 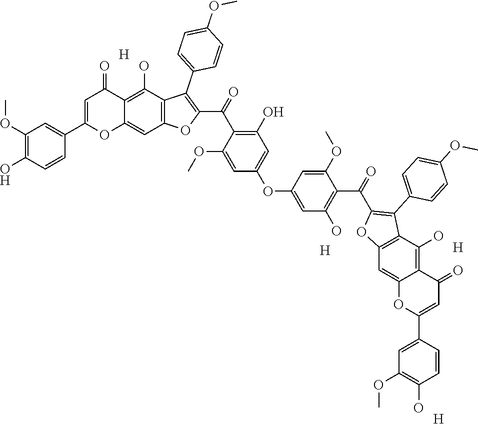 |
| Jacarelhyperol A | 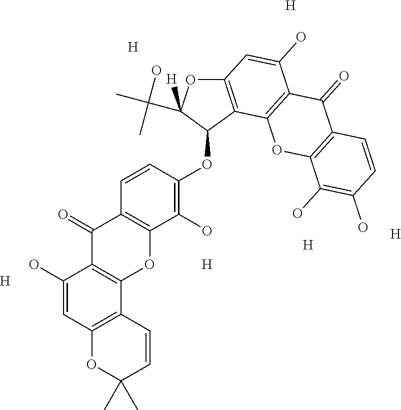 |
| Strychnogucine C | 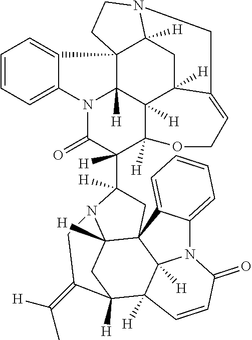 |
| Bisisodiospyrin | 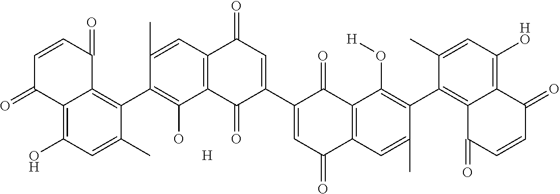 |

### Examples of Pharmaceutically Acceptable Salts

Examples of pharmaceutically acceptable salts of the compounds of Table 1 and, where appropriate, Table 2, include salts derived from suitable inorganic and organic acids, and suitable inorganic and organic bases. Examples of pharmaceutically acceptable acid addition salts are salts of an amino group formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid or by using other methods used in the art, such as ion exchange. Other pharmaceutically acceptable acid addition salts include adipate, alginate, ascorbate, aspartate, benzenesulfonate, benzoate, bisulfate, borate, butyrate, camphorate, camphorsulfonate, cinnamate, citrate, cyclopentanepropionate, digluconate, dodecylsulfate, ethanesulfonate, formate, fumarate, glucoheptonate, glycerophosphate, gluconate, glutarate, glycolate, hemisulfate, heptanoate, hexanoate, hydroiodide, hydroxybenzoate, 2-hydroxy-ethanesulfonate, hydroxymaleate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 2-phenoxybenzoate, phenylacetate, 3-phenylpropionate, phosphate, pivalate, propionate, pyruvate, salicylate, stearate, succinate, sulfate, tartrate, thiocyanate, p-toluenesulfonate, undecanoate, valerate salts, and the like.

Either the mono-, di- or tri-acid salts can be formed, and such salts can exist in either a hydrated, solvated or substantially anhydrous form.

Salts derived from appropriate bases include salts derived from inorganic bases, such as alkali metal, alkaline earth metal, and ammonium bases, and salts derived from aliphatic, alicyclic or aromatic organic amines, such as methylamine, trimethylamine and picoline, or N+((C1-C4)alkyl)4 salts. Representative alkali or alkaline earth metal salts include sodium, lithium, potassium, calcium, magnesium, barium and the like. Further pharmaceutically acceptable salts include, when appropriate, nontoxic ammonium, quaternary ammonium, and amine cations formed using counterions such as halide, hydroxide, carboxyl, sulfate, phosphate, nitrate, lower alkyl sulfonate and aryl sulfonate.

### Pharmaceutical Compositions

The pharmaceutical compositions described herein comprise a therapeutically effective amount of a compound of Table 1, or a pharmaceutically acceptable salt thereof, formulated together with one or more pharmaceutically acceptable carriers or excipients. The pharmaceutical compositions may be administered orally, parenterally, by inhalation spray, topically or via an implanted reservoir.

The term parenteral as used herein includes subcutaneous, intracutaneous, intravenous, intramuscular, intraarticular, intra-arterial, intrasynovial, intrasternal, intrathecal, intralesional and intracranial injection or infusion techniques. Liquid formulations forms for oral administration include pharmaceutically acceptable emulsions, microemulsions, solutions, suspensions, syrups and elixirs.

Liquid dosage forms for oral administration include pharmaceutically acceptable emulsions, microemulsions, solutions, suspensions, syrups, and elixirs. Liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions, may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectable.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Compositions suitable for buccal or sublingual administration include tablets, lozenges and pastilles, wherein the active ingredient is formulated with a carrier such as sugar and acacia, tragacanth, or gelatin and glycerin.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention. The ointments, pastes, creams and gels may contain, in addition to an active compound, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons. Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

For pulmonary delivery, a therapeutic composition is formulated and administered to the patient in solid or liquid particulate form by direct administration e.g., inhalation into the respiratory system. Solid or liquid particulate forms of the active compound prepared for practicing the present invention include particles of respirable size: that is, particles of a size sufficiently small to pass through the mouth and larynx upon inhalation and into the bronchi and alveoli of the lungs. Delivery of aerosolized therapeutics, particularly aerosolized antibiotics, is known in the art (see, for example U.S. Pat. No. 5,767,068 to Van Devanter et al., U.S. Pat. No. 5,508,269 to Smith et al., WO 98/43650 by Montgomery, U.S. Pat. No. 9,956,360 to Germinario et al., U.S. Pat. Pub. No. 2020/0170301 A1, and PCT Publication No. WO 2020/072478 A1, all of which are incorporated herein by reference).

### Therapeutic Methods

In the methods described herein, a viral infection or condition is treated in a subject such as a human or another animal by administering to the subject a therapeutically effective amount of a compound or composition described herein, in such amounts and for such time as is necessary to achieve the desired result. An effective amount of a compound described herein may range from about 0.01 mg/kg to about 500 mg/kg, e.g., from about 0.01 to about 50 mg/kg, from 0.1 to 50 mg/kg, or from 0.1 to 25 mg/kg body weight. Effective amounts or doses will vary depending on route of administration, as well as the possibility of co-usage with other agents.

The total daily dose of the compounds administered to a human or other animal in single or in divided doses can be in separate amounts. Single dose compositions may contain such amounts or submultiples thereof to make up the daily dose. Treatment regimens for the compounds and methods described herein may comprise administration to a patient in need of such treatment from about 10 mg to about 1000 mg, or in some instances more than 1000 mg, of the compound(s) per day in single or multiple doses.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

### Combination Therapies

In certain embodiments, the methods described herein comprise administering a combination of a compound described herein and one or more additional therapeutic or prophylactic agents. In some embodiments, the compound and the additional agent are co-formulated. In another embodiment, the compound and the additional therapeutic agent are co-administered but in different formulations.

In combination therapies, typically both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition. The said “additional therapeutic or prophylactic agents” may include but are not limited to, immune therapies (e.g. interferon), therapeutic vaccines, anti-inflammatory agents such as angiotensin-converting enzyme 2 (ACE 2) inhibitors, corticosteroids or NSAIDs, bronchodilators such as beta-2 adrenergic agonists and xanthines (e.g. theophylline), mucolytic agents, anti-muscarinics, anti-leukotrienes, inhibitors of cell adhesion (e.g. ICAM antagonists), cytokine antagonists, lung surfactants and/or antimicrobial and anti-viral agents.

TABLE 2
Additional (Second) Therapeutic Agents

| | Chemical | | |
|---|---|---|---|
| Name of Therapeutic | Name/Formula | Synonym | Class |
| Rintatolimod | C₂₈H₄₀N₉O₂₅P₃ | | Antiviral |
| (Ampligen) | | | |
| Chloroquine, | C₁₈H₃₂ClN₃ | | Antiviral |
| Hydroxychloroquine | (Chloroquine), | | |
| | C₁₈H₂₈ClN₃O | | |
| | (Hydroxychloroquine) | | |
| Danoprevir | C35H46FN5O9S | | Antiviral |
| EIDD-2801 | C13H19N3O7 | | Antiviral |
| ENU200 | n/a | | Antiviral |
| Elsulfavirine | C₂₄H₁₇BrCl₂FN₃O₅S | | Antiviral |
| Favipiravir | C₅H₄FN₃O₂ | | Antiviral |
| Galidesivir | C₁₁H₁₅N₅O₃ | | Antiviral |
| Lopinavir/ritonavir | C₇₄H₉₆N₁₀O₁₀S₂ | | Antiviral |
| Merimepodib | C₂₃H₂₄N₄O₆ | | Antiviral |
| Nitazoxanide | C₁₂H₉N₃O₅S | | Antiviral |
| OT-101 | n/a | | Antiviral |
| Oxypurinol | C₅H₄N₄O₂ | | Antiviral |
| Remdesivir | C₂₇H₃₅N₆O₈P | | Antiviral |
| Ribavirin | C₈H₁₂N₄O₅ | | Antiviral |
| Acalabrutinib | C₂₆H₂₃N₇O₂ | 1420477-60-6 | Other |
| | | ACP-196 | |
| | | Calquence | |
| | | UNII-I42748ELQW | |
| ACE-MAB (ACE | | | Other |
| monoclonal antibody) | | | |
| ATYR1923A | | | Other |
| Aviptadil (RLF-100) | C₁₄₇H₂₃₇N₄₃O₄₃S | invicorp | Other |
| | | Vasoactive intestinal | |
| | | octacosapeptide | |
| | | Aviptadil [INN:BAN] | |
| | | L-Histidyl-L-seryl-L- | |
| | | aspartyl-L-alanyl-L- | |
| | | valyl-L-phenylalanyl-L- | |
| | | threonyl-L-aspartyl-L- | |
| | | asparaginyl-L-tyrosyl-L- | |
| | | threonyl-L-arginyl-L- | |
| | | leucyl-L-arginyl-L-lysyl- | |
| | | L-glutaminyl-L- | |
| | | methionyl-L-alanyl-L- | |
| | | valyl-L-lysyl-L-lysyl-L- | |
| | | tyrosyl-L-leucyl-L- | |
| | | asparaginyl-L-seryl-L- | |
| | | isoleucyl-L-leucyl-L- | |
| | | asparagine | |
| Baricitinib | C₁₆H₁₇N₇O₂S | 1187594-09-7 | Other |
| | | INCB028050 | |
| | | LY3009104 | |
| | | INCB 028050 | |
| Brensocatib | C₂₃H₂₄N₄O₄ | AZD7986 | Other |
| | | 1802148-05-5 | |
| | | AZD-7986 | |
| | | UNII-25CG88L0BB | |
| | | 25CG88L0BB | |
| Brilacidin | C₄₀H₅₀F₆N₁₄O₆ | 1224095-98-0 | Other |
| | | UNII-I1679X069H | |
| | | I1679X069H | |
| | | PMX30063 | |
| Bucillamine | C₇H₁₃NO₃S₂ | 65002-17-7 | Other |
| | | Tiobutarit | |
| | | Rimatil | |
| | | N-(2-Mercapto-2- | |
| | | methylpropionyl)-L- | |
| | | cysteine | |
| Centhaquine | C₂₂H₂₅N₃ | Centhaquin | Other |
| | | 57961-90-7 | |
| | | Compound 7173 | |
| | | UNII-QD4VI0J9T5 | |
| Convalescent plasma | | | Other |
| Dapagliflozin | C₂₁H₂₅ClO₆ | 461432-26-8 | Other |
| | | BMS-512148 | |
| | | Forxiga | |
| | | Farxiga | |
| DAS181 | | | Other |
| gammaCore | | | Other |
| CD24Fc | | | Other |
| CM4620-IE | | | Other |
| Famotidine | C₈H₁₅N₇O₂S₃ | 76824-35-6 | Other |
| | | Pepcid AC | |
| | | Pepcidine | |
| | | Pepcid RPD | |
| Gimsilumab | | | Other |
| HB-adMSC | | | Other |
| Ifenprodil | C₂₁H₂₇NO₂ | 23210-56-2 | Other |
| | | ifenprodil tartrate | |
| | | Vadilex | |
| | | Dilvax | |
| Inhaled nitric oxide | NO | | Other |
| Ivermectin | C₄₈H₇₄O₁₄ | Ivermectin B1a | Other |
| | | Dihydroavermectin B1a | |
| | | 70288-86-7 | |
| | | 22,23-Dihydroavermectin B1a | |
| LB1148 | C₈H₁₅NO₂ | tranexamic acid | Other |
| | | 1197-18-8 | |
| | | Cyklokapron | |
| | | trans-4- | |
| | | (Aminomethyl)cyclohexanecarboxylic acid | |
| | | Tranexamsaeure | |
| LEAPS peptides | | | Other |
| LY3127804 | | | Other |
| Mavrilimumab | | | Other |
| Multi-antibody cocktail | | | Other |
| therapy | | | |
| MultiStem therapy | | | Other |
| Pacritinib | C₂₈H₃₂N₄O₃ | 937272-79-2 | Other |
| | | Pacritinib (SB1518) | |
| | | SB1518 | |
| | | SB-1518 | |
| Remestemcel-L | | | Other |
| Ruxolitinib | C₁₇H₁₈N₆ | 941678-49-5 | Other |
| | | INCB018424 | |
| | | Ruxolitinib | |
| | | (INCB018424) | |
| | | INCB-018424 | |
| Sargramostim | | | Other |
| Sarilumab | | | Other |
| Siltuximab | | | Other |
| ST266 | | | Other |
| Tocilizumab | | | Other |
| TAK-888 | | | Other |
| Tradipitant | C₂₈H₁₆ClF₆N₅O | 622370-35-8 | Other |
| | | LY-686017 | |
| | | VLY-686 | |
| | | UNII-NY0COC51FI | |
| TD-0903 | | | Other |
| TZLS-501 | | | Other |
| Vazegepant | | | Other |
| CYNK-001 | | | Other |
| I-Mab's therapy | | | Other |
| SAB-185 | | | Antiviral |
| Dexamethasone | C₂₂H₂₉FO₅ | 50-02-2 | Other |
| | | Decadron | |
| | | Dexamethazone | |
| | | Maxidex | |
| Favilavir | | | Antiviral |
| BPI-002 | | | Other |
| TJM2 | | | Other |
| AT-100 (rhSP-D) | | | Other |
| Leronlimab | | | Other |

Where appropriate, the agents of Table 2 can be formulated as a pharmaceutically acceptable salt, as described herein.

### Synthetic Methods

Methods of synthesizing the compounds herein will be evident to those of ordinary skill in the art. Synthetic chemistry transformations and protecting group methodologies useful in synthesizing the compounds described herein are known in the art and include, for example, those such as described in Larock (Ed.), Comprehensive Organic Transformations, 4 Volume Set: A Guide to Functional Group Preparations, Wiley 3d edition (2018); Wuts, Greene's Protective Groups in Organic Synthesis, Wiley 5th edition (2014); Ho, Fiesers' Reagents for Organic Synthesis (Book 29), Wiley (2019); and L. Paquette (Ed.), Encyclopedia of Reagents for Organic Synthesis, Wiley (2009). The compounds of this invention may be modified by appending appropriate functionalities to enhance selective biological properties. Such modifications are known in the art and may include those which increase biological penetration into a given biological system (e.g., blood, lymphatic system, central nervous system), increase oral availability, increase solubility to allow administration by injection, alter metabolism and alter rate of excretion.

### Cell and Vesicle-Based Carriers

A compound or composition described herein can be administered in a vesicle or other membrane-based carrier.

In embodiments, a compound or composition described herein is administered in or via a cell, vesicle or other membrane-based carrier. In one embodiment, the compound or composition can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for the compound or composition described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid—polymer nanoparticles (PLNs), a new type of carrier that combines liposomes and polymers, may also be employed. These nanoparticles possess the complementary advantages of PNPs and liposomes. A PLN is composed of a core—shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Additional non-limiting examples of carriers include carbohydrate carriers (e.g., an anhydride-modified phytoglycogen or glycogen-type material), protein carriers (e.g., a protein covalently linked to the circular polyribonucleotide), or cationic carriers (e.g., a cationic lipopolymer or transfection reagent). Non-limiting examples of carbohydrate carriers include phtoglycogen octenyl succinate, phytoglycogen beta-dextrin, and anhydride-modified phytoglycogen beta-dextrin. Non-limiting examples of cationic carriers include lipofectamine, polyethylenimine, poly(trimethylenimine), poly(tetramethylenimine), polypropylenimine, aminoglycoside-polyamine, dideoxy-diamino-b-cyclodextrin, spermine, spermidine, poly(2-dimethylamino)ethyl methacrylate, poly(lysine), poly(histidine), poly(arginine), cationized gelatin, dendrimers, chitosan, 1,2-Dioleoyl-3-Trimethylammonium-Propane(DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), 3B-[N—(N\N′-Dimethylaminoethane)-carbamoyl]Cholesterol Hydrochloride (DC-Cholesterol HC 1), diheptadecylamidoglycyl spermidine (DOGS), N,N-distearyl-N,N-dimethylammonium bromide (DDAB), N-(1,2-dimyristyloxyprop-3-yl)-N,N-dimethyl-N-hydroxyethyl ammonium bromide (DMRIE), and N,N-dioleyl-N,N-dimethylammonium chloride (DODAC). Non-limiting examples of protein carriers include human serum albumin (HSA), low-density lipoprotein (LDL), high-density lipoprotein (HDL), or globulin.

Exosomes can also be used as drug delivery vehicles for a compound or composition described herein. For a review, see Ha et al. July 2016. Acta Pharmaceutica Sinica B. Volume 6, Issue 4, Pages 287-296; https://doi.org/10.1016/j.apsb.2016.02.001.

Ex vivo differentiated red blood cells can also be used as a carrier for a compound or composition described herein. See, e.g., WO2015073587; WO2017123646; WO2017123644; WO2018102740; wO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver the compound or composition described herein.

Virosomes and virus-like particles (VLPs) can also be used as carriers to deliver a compound or composition described herein to target cells.

Plant nanovesicles and plant messenger packs (P1VIPs), e.g., as described in WO2011097480, WO2013070324, WO2017004526, or WO2020041784 can also be used as carriers to deliver the compound or composition described herein.

### EXEMPLIFICATION

### Example 1: Preparation of Compounds

This example describes the preparation of compounds described herein.

### Example 1.1: Hypericin

Hypericin is purchased from Sigma Aldrich (SKU 56690). Hypericin may be extracted from Hypericum perforatum using ethanol and high pressure according to the protocol in [Cossuta et al. (2011) Journal of Food Process Engineering, Volume 35, Issue 2 p. 222-235 DOI: 10.1111/j.1745-4530.2010.00583.x].

### Example 1.2: Fagopyrine

Fagopyrine is extracted from dried buckwheat herb according to the protocol in [Hinneburg and Neubert (2005) J. Agric. Food Chem., Volume 53, Issue 1, p. 3-7 https://doi.org/10.1021/jf049118f].

### Example 1.3: Protohypericin

Protohypericin is obtained by subsequent oxidation of emodin bianthrone with oxygen in methanol containing triethylamine according to the protocol in [Barnard, D. L. et al (1992) Antiviral Research. Volume 17: p 63-77. PMID: 1310583].

### Example 1.4: Pseudohypericin

Pseudohypericin is purchased from Sigma Aldrich (CAS Number 55954-61-5). In brief, Pseudohypericin may be isolated from Hypericum perforatum by purification using hydro-alcoholic dried extracts and column chromatography, then confirmed using liquid chromatography-mass spectrometry according to the protocol in [Karioti, et al (2009), J. Sep. Science, volume 32: p. 1374-1382. https://doi.org/10.1002/jssc.200800700].

### Example 1.5: Artonin A

Artonin A is extracted from the dried root bark or Artocarpus heterophyllus in n-hexane, benzene, and acetone, followed by column chromatography and filtration according to the protocol in [Hano Y, et al (1989) Heterocycles Vol. 29: P. 1447-1453. DOI: 10.3987/COM-89-5019].

### Example 1.6: Trisjuglone

Trisjuglone is extracted from the dried bark of Juglans regia L. (common walnut tree) in hexane, chloroform, ethyl acetate, and methanol, concentrated, and isolated with silica gel column chromatography according to the protocol in [Strugstad, M., & Despotovski, S. (2013). Journal of Ecosystems and Management, Volume 13(3) p. 1-16].

### Example 1.7: Casuarictin

Casuarictin is purchased from Nacalai USA. Casuarictin also be isolated from clove or mangrove following the protocol in [Rodrigues et al., Mar Drugs. (2019) July; Volume 17(7): p. 403.]

### Example 1.8: Tellimagrandin I

Tellimagrandin I is purchased from Nacalai USA.

### Example 1.9: Alnusiin

Alnusiin is isolated from Alnus sieboldiana following the protocol in Hirokane et al., A unified strategy for the synthesis of highly oxygenated diaryl ethers featured in ellagitannins. [Hirokane et. Al (2014) Nature Communications Vol 5: No. 3478. DOI: 10.1038/ncomms4478.]

### Example 1.10: Vescalagin

Vescalagin is purchased from Sigma Aldrich.

### Example 1.11: Punicalagin

Punicalagin is purchased from Sigma Aldrich.

### Example 1.12: Theaflavin

Theaflavin is purchased from Sigma Aldrich. Described in US 2008/0254190 A1, generally, extracted with Urea from tea, and purified with HPLC.

### Example 1.13: Theaflavin-3-Fallate

Theaflavin-3-gallate is purchased from Sigma Aldrich. Described in US 2008/0254190 A1, generally, extracted with Urea from tea, and purified with HPLC.

### Example 1.14: Hinokiflavone

Hinokiflavone is synthesized according to the protocol [Koichi Nakazawa. (1967), Tetrahedron Letters, Volume 8, Issue 51, Pages 5223-5225, https://doi.org/10.1016/S0040-4039(01)89648-9.].

### Example 1.15: Ginkgetin

Ginkgetin is purchased from Sigma Aldrich.

### Example 1.16: Podocarpusflavone A

Podocarpusflavone A is purchased from LifeTein.

### Example 1.17: Sequoiaflavone

Sequoiaflavone is extracted from Ouretea ferruginea following the protocol in [Fidelis Q C, et al. (2012) Molecules. Vol. 17(7): p. 7989-8000. doi: 10.3390/molecules17077989.]. Briefly, ground leaves are extracted with methanol and purified by column chromatography.

### Example 1.18: Sotetsuflavone

Sotetsuflavone is purchased from MedChemExpress.

### Example 1.19: Taiwanhomoflavone A

Taiwanhomoflavone A is purchased from BioCrick.

### Example 1.20: Amentoflavone

Amentoflavone is purchased from Sigma Aldrich.

### Example 1.21: Bilobetin

Bilobetin is purchased from Sigma Aldrich.

### Example 1.22: Smitilbin

Smitilbin is synthesized according to the protocol in U.S. Pat. No. 6,706,865 B2. Briefly, protected catechins are reacted with sugar derivatives.

### Example 1.23: Diathin F

Diathin F is synthesized by cyclic peptide synthesis.

### Example 1.24: 2-Phloroeckol

2-Phloroeckol is extracted from E. stolonifera according to the protocol in [Yoon, et al (2008). Fisheries Sci. Vol. 74, p. 200, https://doi.org/10.1111/j.1444-2906.2007.01511.x]. Briefly, E. stolonifera is ground and extracted with ethanol and then separated by hexane-ethyl acetate extraction. The ethyl acetate fraction is dried and purified with HPLC to yield 2-phloroeckol.

### Example 1.25: Ergotamine

Ergotamine is purchased from Sigma Aldrich.

### Example 1.26: Bismahanine

Bismahanine is extracted from Murraya koenigii leaves according to the protocol in [Tachibana et al, (2003) J. Agric Food Chem, vol. 51 p. 6461-6467, https://doi.org/10.1021/jf034700+]. Briefly, ground leaves are extracted with dichloromethane, partitioned with ethyl acetate, and run on a silicon gel column to yield Bismahanine.

### Example 1.27: Lactucain C

Lactucain C is extracted from Lacutuca Indica according to the methods in Hou et al, J. Nat Prod, 2003. Briefly, plants are extracted with acetone, dried, solvent extracted with n-butanol, and purified with HPLC.

### Example 1.28: Jugnaphthalenoside C

Jugnaphthalenoside C is extracted from Juglans cathayensis according to the methods in Sun et al, Chem Pharm Bull, 2012. Briefly root bark is extracted with ethanol, followed by repartitioning in n-butanol and purification with HPLC to yield Jugnapthalenoside C.

### Example 1.29: Usambarensine

Usambarensine is extracted from S. usambarensis according to the methods in [Frederich Met. Al., (1999), Antimicrob Agents Chemother. Vol. 43(9), p. 2328-31. doi:10.1128/AAC.43.9.2328.]. Briefly, root bark is powdered, extracted with ethanol and purified with HPLC.

### Example 1.30: Grandione

Grandione is extracted from betel nut according of the protocol in Kusumoto et al, Phytother. Res., (1995), Vol. 9, p. 180-184, https://doi.org/10.1002/ptr.2650090305. Briefly, ground betel nut is extracted with acetone, then extracted in hexane and ethyl acetate. The ethyl acetate fraction is purified by HPLC to yield Arecatann.

### Example 1.31: Neoacrimarine H

Neoacrimarine H is extracted from the root of Citrus paradisi according to the methods in Takemura et al, Chem Pharm Bullet., (1998), Vol. 46, p. 1518-1521, https://doi.org/10.1248/cpb.46.1518. Briefly, the root is ground and extracted with acetone and purified with HPLC to yield Neoacrimarine H.

### Example 2: Anti-Viral Activity Assay

Vero E6 cells are acquired from ATCC and plated according to the manufacturer's instructions. Growing Vero E6 cells are plated into a 96 well plate and pretreated for 1-24 hours with 0, 0.01, 0.1, 1, 10, 100, 1000, and 10000 nmol of a compound prepared as described in Example 1 and dissolved in DMSO, water or PBS. Cytotoxicity of the compound alone is assessed using Cell Titer Glo after 6, 12, 24, 48, and 72 hours.

After validation of optimal concentration range of the compound pretreatment, SARS-CoV-2 is then applied to the pre-treated Vero E6 cells at a multiplicity of infection of 0.01, 0.05, 0.1, 0.5, 1, 10 plus a no virus control. At 2 days post infection, cells are fixed using 10% formalin. Fixed cells are subjected to immunofluorescent staining using a primary antibody directed against the SARS-CoV-2 nucleoprotein. Cell nuclei are stained with DAPI. Infection rates are determined by quantification of SARS-CoV-2-positive cells. Cell viability is determined by counting DAPI-positive cells and comparing to control.

### Example 3: Treatment of SARS-CoV-2-Infection with Food-Derived Compounds

Numerous food-derived compounds were screened for the ability to decrease viral replication of the novel SARS-CoV-2 coronavirus. Disclosed herein are certain compounds that showed anti-viral activity.

Example 3 demonstrates the ability of compounds disclosed herein (Table 3) to decrease viral replication of the novel SARS-CoV-2 coronavirus in infected Vero E6 primate cells and to inhibit the viral proteases in direct enzyme assays.

TABLE 3
List of food-derived compounds

| Compound Name | Supplier | Catalogue Number |
|---|---|---|
| Amentoflavone | Sigma Aldrich | PHL80351-10MG |
| Bilobetin | Sigma Aldrich | PHL83840-5MG |
| Delphinidin 3,5-Diglucoside | Sigma Aldrich | PHL89626-5MG |
| (Delphinidin) | | |
| Dioscin | Sigma Aldrich | SMB00576-25MG |
| Ergotaminine | Sigma Aldrich | 1241550-100MG |
| Ginkgetin | Sigma Aldrich | PHL83501-10MG |
| Hypericin | Sigma Aldrich | PHL89226-10MG |
| Miquelianin/Quercetin | Sigma Aldrich | PHL80349-10MG |
| 3-glucuronide | | |
| Procyanidin B2 | Sigma Aldrich | PHL89552-10MG |
| Punicalagin | Sigma Aldrich | P0023-10MG |
| Robinin | Sigma Aldrich | PHL83246-10MG |
| Rutin | Sigma Aldrich | PHL89270-50MG |
| Theaflavin | Sigma Aldrich | PHL83341-10MG |
| Theaflavin 3-Gallate | Sigma Aldrich | PHL83342-10MG |
| Tiliroside | Sigma Aldrich | PHL89809-10MG |

### a) Effect of Food-Derived Compounds on Vero E6 Cell Viability

### Dose:

All compounds were dissolved in DMSO to a stock concentration of 10 mM. Vero E6 cells were treated with compounds at a final concentration of 1 nM, 10 nM, 100 nM, 1 μM, 10 μM, 100 μM and 1 mM, or the vehicle control (DMSO), in cell culture medium.

### Experimental Design and Results:

Vero E6 cells were obtained from the ATCC (VERO C1008 [Vero 76, clone E6, Vero E6] (ATCC® CRL-1586™)) and grown and maintained according to the supplier's instructions. Vero E6 cell line derived from African green monkey kidney epithelial cells was used as in vitro SARS-CoV-2 infection model. Cells at 70-80% confluency were harvested, counted and seeded in 96-well culture treated well plate at a seeding density of 10,000 cells per well in cell culture medium. 24 hours after seeding, cells were incubated with one of the compounds from Table 3 at concentrations of 1 nM, 10 nM, 100 nM, 1 μM, 10 μM, 100 μM and 1 mM, or the vehicle only control (DMSO, Sigma, D2650), in cell culture medium for 48 hours at 37° C. These experiments were performed in triplicate.

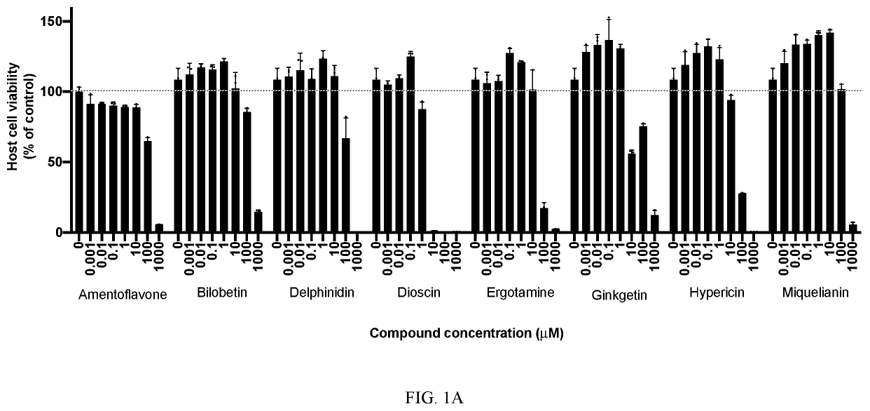

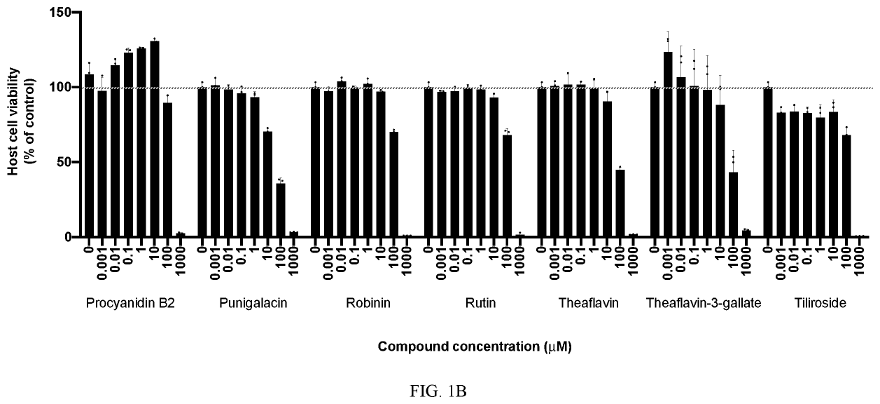

To determine the effect of the compounds on the viability of Vero E6 cells, a CellTiterGlo (Promega) luminescent cell viability assay was performed according to the manufacturer's instruction, and luminescence intensities were measured on a SpectraMax microplate reader. Luminescence data was normalized to DMSO control samples, and percent viability was plotted against the compound concentration (FIGS. 1A-B).

For Amentoflavone, Bilobetin, Delphinidin, Ergotamine, Procyanidin B2, Robinin, Rutin, Theaflavin, and Theaflavin-3-gallate, concentrations of less than or equal to 10 μM did not significantly impact Vero E6 cell viability compared to the DMSO treated control, with toxicity observed at and above 100 μM.

For Tiliroside, concentrations of less than or equal to 10 μM only mildly impacted Vero E6 cell viability compared to the DMSO treated control, with toxicity observed at and above 100 μM.

For Dioscin, concentrations of less than or equal to 0.1 μM did not significantly impact Vero E6 cell viability compared to the DMSO treated control, with toxicity observed at and above 1 μM.

For Ginkgetin, Hypericin, Punicalagin concentrations of less than or equal to 1 μM did not significantly impact Vero E6 cell viability compared to the DMSO treated control, with toxicity observed at and above 10 μM.

For Miquelianin, concentrations of less than or equal to 100 μM did not significantly impact Vero E6 cell viability compared to the DMSO treated control, with toxicity observed at 1000 μM.

### b) Effect of Compound Pre-Treatment on Viral Load in SARS-Cov-2 Infected Vero E6 Cells

To next determine the effect of compound pre-treatment on SARS-CoV-2 viral replication, Vero E6 cells were seeded at 10,000 cells/well in 96-well plates and were pretreated for 1 hour with the respective compound at final concentration of 1 nM, 10 nM, 100 nM, 1 μM, 10 μM, and 100 μM, or the vehicle only control, in triplicate.

SARS-CoV-2 virus (isolate USA_WA1/2020, kindly provided by CDC's Principal Investigator Natalie Thornburg and the World Reference Center for Emerging Viruses and Arboviruses (WRCEVA)”) was then directly added to the wells at a multiplicity of infection (MOI) of 0.05, and incubated in the presence of the compounds for 48 hours at 37° C. in a BSL-4 laboratory. After 48 hours, cells were fixed using 10% formalin. Fixed cells were subjected to immunofluorescence using a primary antibody directed against the SARS-CoV-2 nucleoprotein (Rockland; 200-401-A50; 1:2000), and a secondary GFP-labeled antibody (Goat anti-Rabbit IgG (H+L) Highly Cross-Adsorbed Secondary Antibody, Alexa Fluor 488 (Thermo Fisher; A-11034; 1:200)). The resulting fluorescence signal was used as a proxy for viral burden. Cell nuclei were stained with DAPI (Sigma; D9542; 1:5000) to quantify the total cell number.

Images were processed using CellProfiler 3.1.9. to identify cell nuclei (DAPI) and presence of SARS-CoV-2-infection (GFP intensity). Each DAPI-positive cell was then classified as “GFP positive” (SARS-CoV-2-infected) or “GFP negative” (non-SARS-CoV-2-infected), with the threshold set based on images of cells unexposed to any SARS-CoV-2 (negative control) and images of virus-infected cells that were untreated with any compound (positive control). A cell is considered infected if both DAPI-positive and GFP-positive staining are observed. If a cell is only DAPI-positive nucleus but GFP-negative, the cell is considered uninfected by the SARS-CoV-2 virus. Infection rates were determined by normalization of SARS-CoV-2-infected cells to the total cell number DAPI-positive cells. Relative levels of infection (“Viral Load”) were further calculated by normalizing the infection rate at each concentration against the vehicle-treated control (DMSO). The number of DAPI-positive cells in each treatment condition was used as an indicator of cell number “Host Cell Number”). A 3-parameter standard curve was fit with the concentrations as x values. The control GFP-positive cells (vehicle control DMSO treated, virus infected) were scaled to 100%. EC50 was defined as the concentration at which the inhibitor achieves 50% response based on the standard curve.

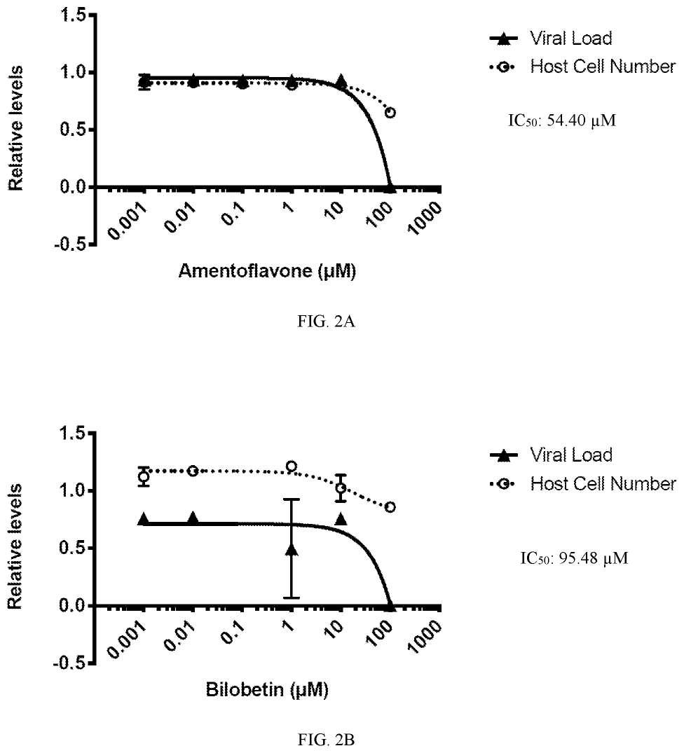

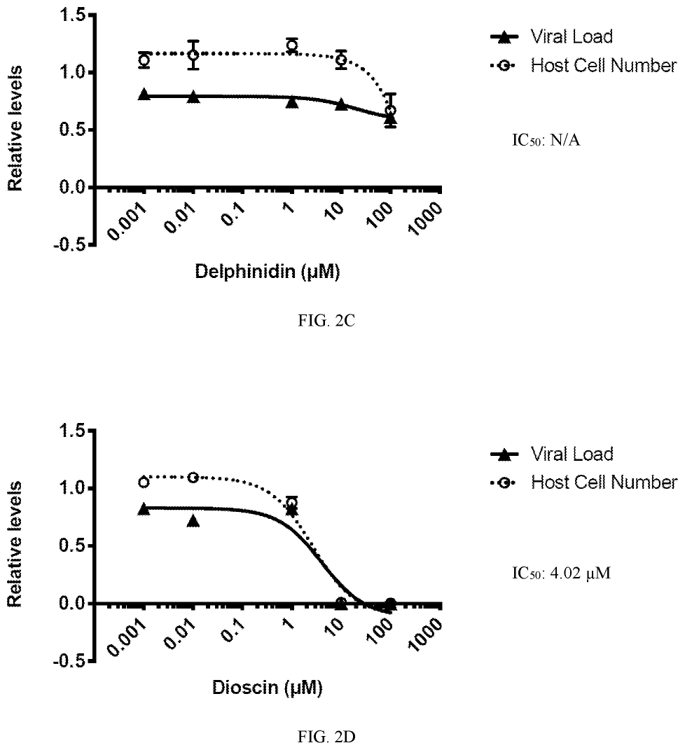

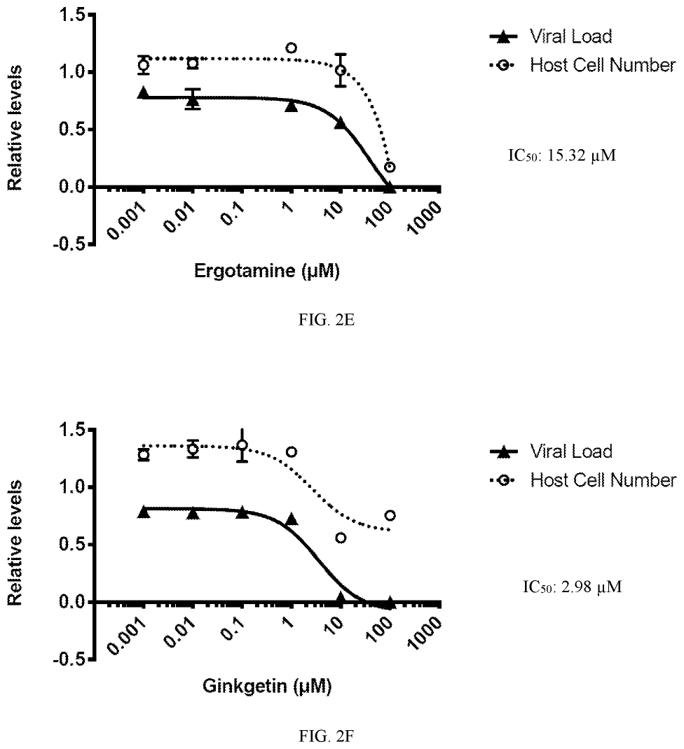

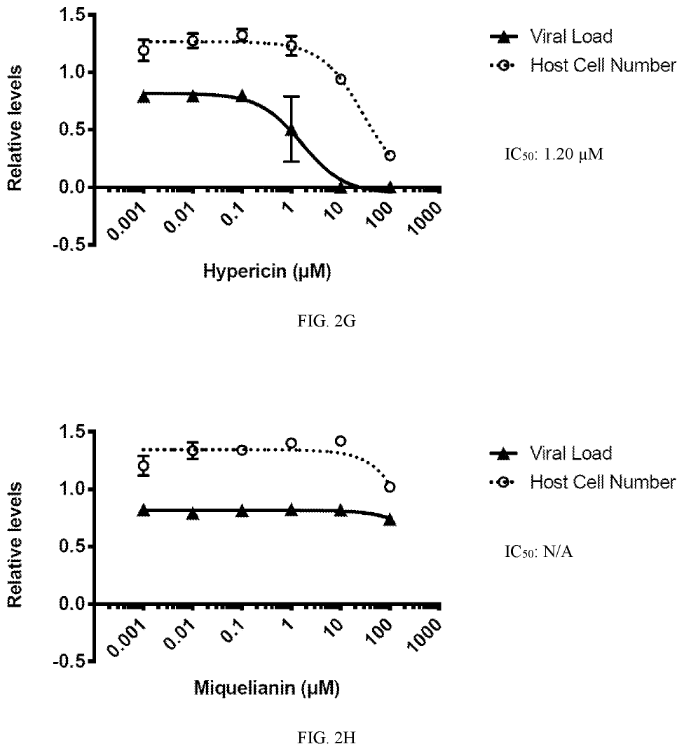

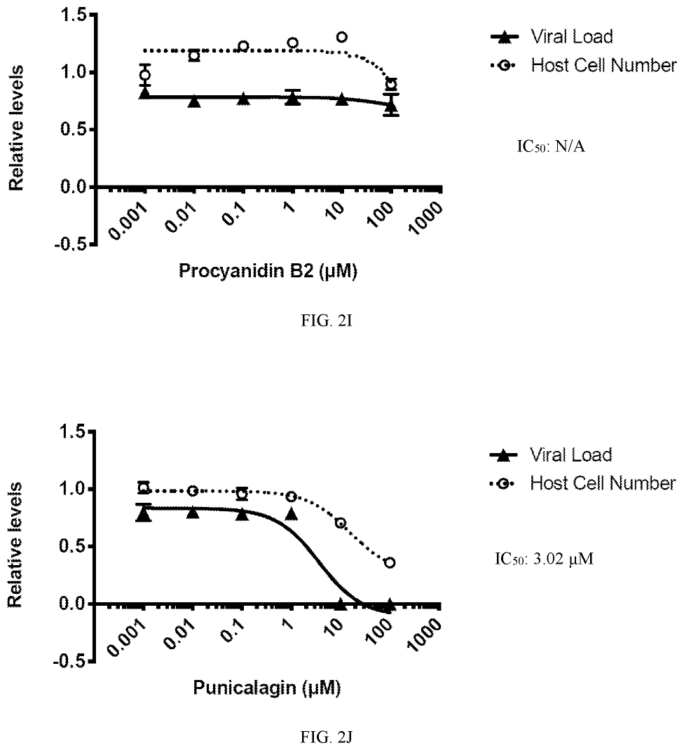

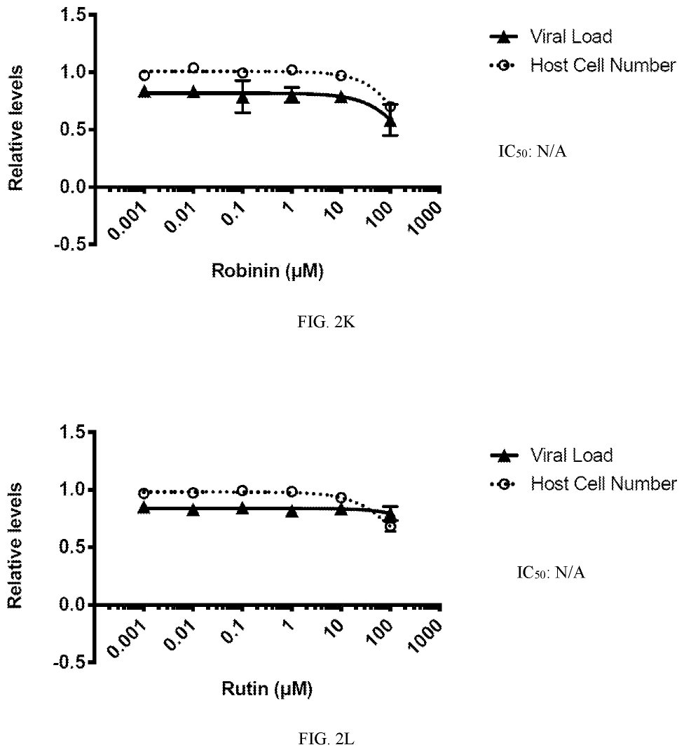

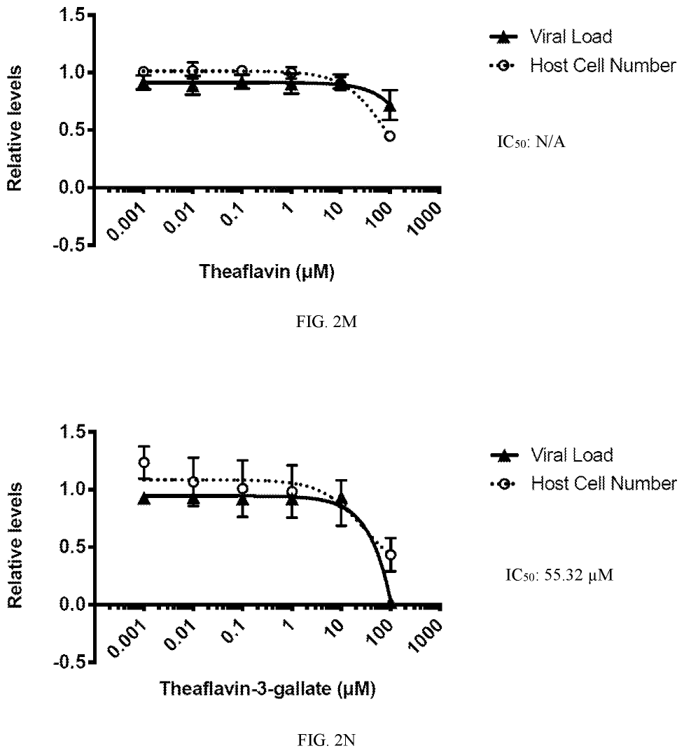

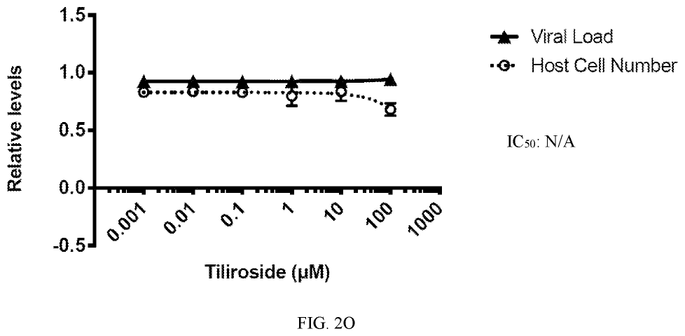

The effect of increasing concentration of the compounds on Viral Load and Host Cell Number may be found in FIGS. 2A-O. Delphinidin, Miquelianin, Procyanidin B2, Robinin, Theaflavin, Rutin, or Tiliroside are examples of a compound that did not show an effect on viral burden nor cell viability, as represented by unchanged Viral Load and Host Cell Number levels across the tested concentrations (FIGS. 2C, H, I, K, L, M, O). Dioscin, displayed a reduction in Viral Load levels, but also a similar reduction in Host Cell Number, indicating toxicity of the compound (FIG. 2D). Pre-treatment with Amentoflavone, Bilobetin, Ergotamine, Ginkgetin, Hypericin, Punicalagin, or Theaflavin-3-gallate however showed a reduction in Viral Load levels at concentrations that did not coincide with loss in Host Cell Number, with a therapeutic index greater than 2 (FIGS. 2A, B, E, F, G, J, N). These results indicate that these compounds can reduce SARS-CoV-2 viral replication in Vero E6 primate cells.

### c) Effect of Compounds on SARS-CoV-2 Main (M) and Papain-Like (PL) Proteases in Direct Enzyme Assays

Given the inhibitory effect on SARS-CoV-2 viral load in vitro, several compounds (Amentoflavone, Theaflavin-3-gallate, Punicalagin) and some of Punicalagin's metabolites (Urolithin A (Sigma Aldrich), Urolithin B (Sigma Aldrich), Ellagic Acid (Sigma Aldrich), and Vescalagin (Sigma Aldrich)) were tested for their ability to inhibit the activity of two isolated SARS CoV-2 proteases in a direct enzyme assay. The main protease (Mpro) is required to cleave viral polyproteins, including those required for viral replication. Inhibition of this protease prevents the virus from replicating marking it as a possible therapeutic target for preventing or treating SARS-CoV-2 infection (Sacco et al. (2020); Coelho et al. (2020)). Similarly, the papain-like protease (PLpro) is required for polyprotein processing and represents an alternative therapeutic target for the treatment of SARS CoV-2 infections (Klemm et al. (2020)).

Mpro Assay: The total reaction volume was 50 μL. Compounds were pre-dispensed into black 384 well plates (Corning) using an Echo 550 acoustic dispenser (Labcyte). All compounds were solubilized in DMSO. The compound volume varied depending on the final concentration and wells were topped up with DMSO using a Multidrop Combi nL reagent dispenser (Thermo Fisher) to provide a final concentration of 2% (1 μL total volume). A Tempest dispenser (Formulatrix) was used to add 30 μL of reaction mixture to give a final concentration of 50 mM HEPES pH 7.5, 5 mM L-glutathione reduced, 0.1 mg/mL BSA and 0.0125 μM Mpro. The compound solution was incubated for 10 minutes at room temperature and then the reaction was initiated by the addition of 19 μL of 25 μM fluorescent peptide substrate using a Multidrop Combi nL reagent dispenser (Thermo Fisher). The plates were placed in a vacuum chamber for 1.5 minutes to remove bubbles and the fluorescence was read every 65 seconds for 14 minutes in a Synergy Neo2 plate reader (Biotek) with an excitation wavelength of 360 nm and an emission wavelength of 460 nm.

PLpro Assay: The total reaction volume was 50 μL. Compounds were pre-dispensed into black 384 well plates (Corning) using an Echo 550 acoustic dispenser (Labcyte). All compounds were solubilized in DMSO. The compound volume varied depending on the final concentration and wells were topped up with DMSO using a Multidrop Combi nL reagent dispenser (Thermo Fisher) to provide a final concentration of 2% (1 μL total volume). A Tempest dispenser (Formulatrix) was used to add 30 μL of reaction mixture to give a final concentration of 50 mM HEPES pH7.5, 5 mM L-glutathione reduced, 0.1 mg/mL BSA and 0.1 μM PLpro. The compound solution was incubated for 10 minutes at room temperature and then the reaction was initiated by the addition of 19 μL of 325 μM fluorescent peptide substrate using a Multidrop Combi nL reagent dispenser (Thermo Fisher). The plates were placed in a vacuum chamber for 1.5 minutes to remove bubbles and the fluorescence was read every 65 seconds for up to 14 minutes in a Synergy Neo2 plate reader (Biotek) with an excitation wavelength of 320 nm and an emission wavelength of 405 nm.

Screening data consists of a 14-minute kinetic read. Slopes were calculated using the data from 0 to 14:05 minutes for PLpro and from 0 to 9:45 minutes for Mpro. Slopes were normalized on each plate using control-based normalization, where:

$$\% activity = ( \frac{S - L}{H - L} ) \times 100 \qquad ( Eq . 1 )$$

Where S=the sample slope

L=the slope of the low activity control

H=the slope of the high activity control

Dose response data was fitted using a four parameter logistic (4PL) non-linear regression model constrained to a maximum response of 1 and a minimum response of 0. The equation used for the 4PL curves was:

$$y = d + ( \frac{a - d}{1 + {( \frac{x}{c} )}^{b}} ) \qquad ( Eq . 2 )$$

Where y=the sample response in relative luminescence units, x=the drug concentration, a=the maximum response for infinite standard concentration, b=−Hill slope, c=inflection point, d=the response at a standard concentration of 0.

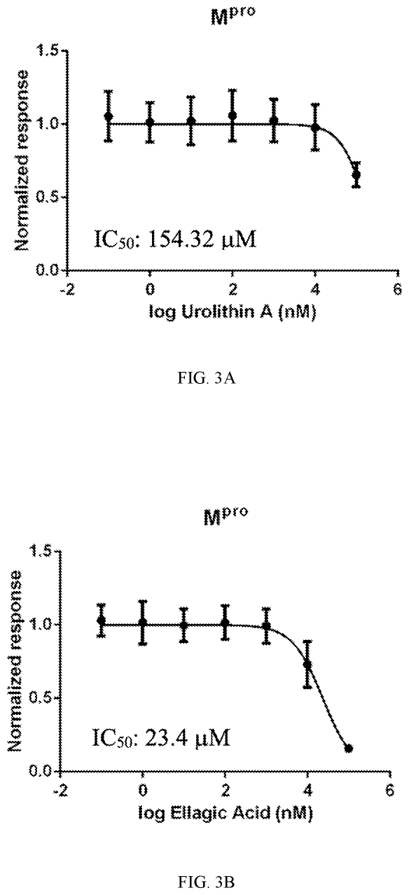

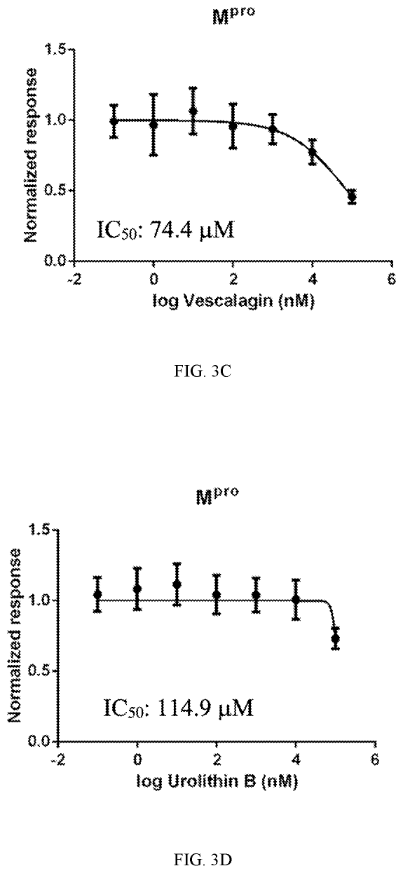

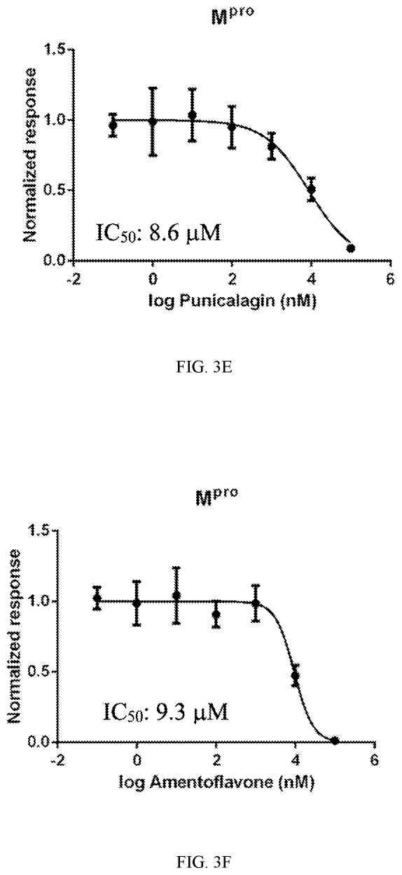

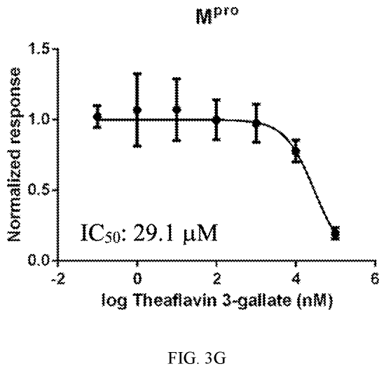

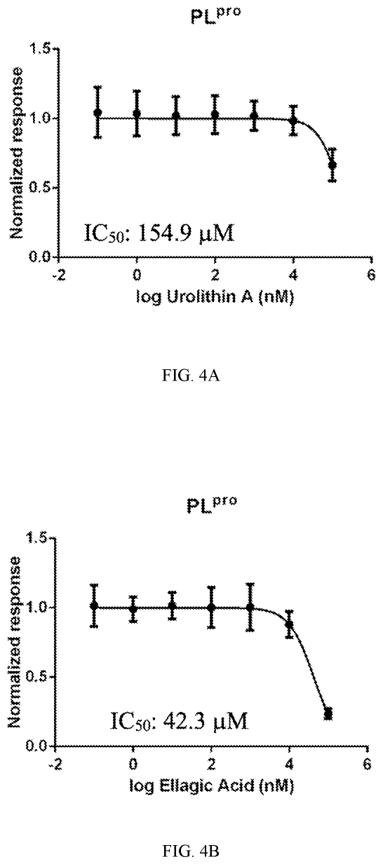

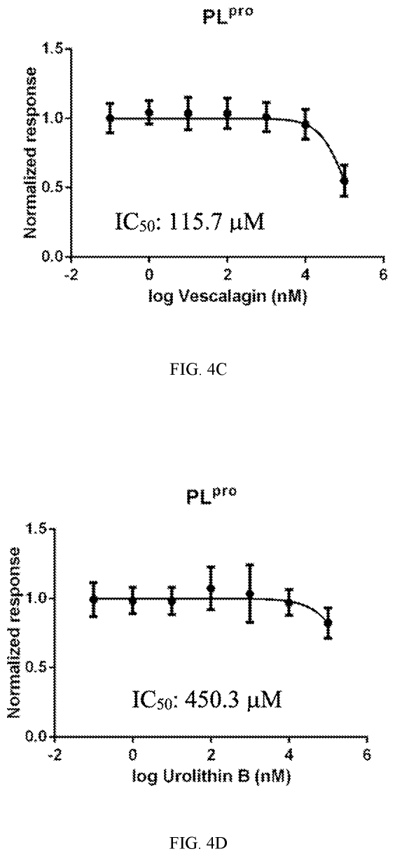

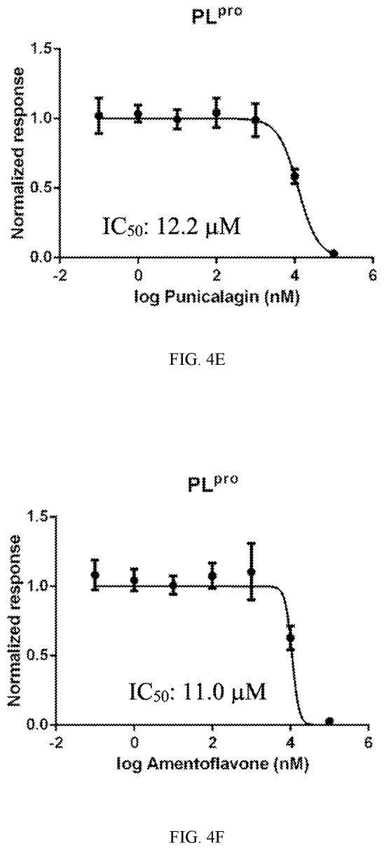

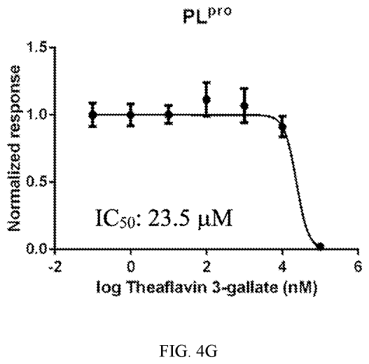

Using these equations, the drug concentration was calculated that results in a 50% reduction in enzyme activity (IC50). Dose response curve were generated for various compounds against the M protease (FIGS. 3A-G). Dose response curve were generated for various compounds against the PL protease (FIGS. 4A-G).

### d) Combined Effect of Food-Derived Compounds and Remdesivir on Host Cell Viability in SARS-CoV-2-Infected Vero E6 Cells

The food-derived compounds were tested in combination with a current antiviral, Remdesivir, against SARS-CoV-2 infection. Vero E6 cells were seeded at 25,000 cells/well in 96-well plates and SARS-CoV-2 was added into the wells at a MOI of 0.01. After 1 hour, compounds were added into the wells at final concentrations of 0.0316 μM, 0.1 μM, 0.316 μM, 1 μM, 3.16 μM, 10 μM and 31.6 μM or the vehicle only control (DMSO), alongside Remdesivir at final concentrations of 0.15 μM, 0.31 μM, 0.62 μM, 1.25 μM, and 2.5 μM or the vehicle only control (DMSO), and incubated at 37° C. After 72 hours of incubation, host cell viability was assessed using CellTiter-Glo® Luminescent Cell Viability Assay (Promega) according to the manufacturer's instructions. Results are normalized to the highest dose of Remdesivir alone (2.5 μM).

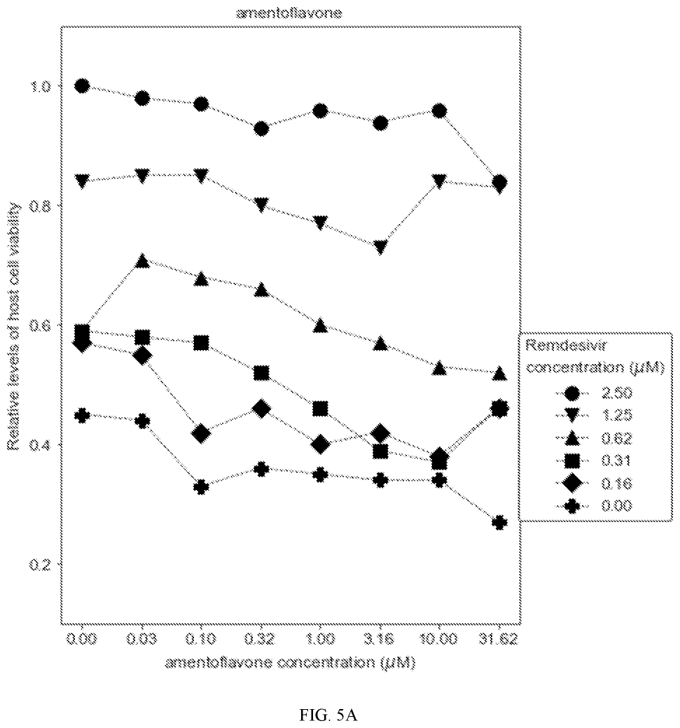

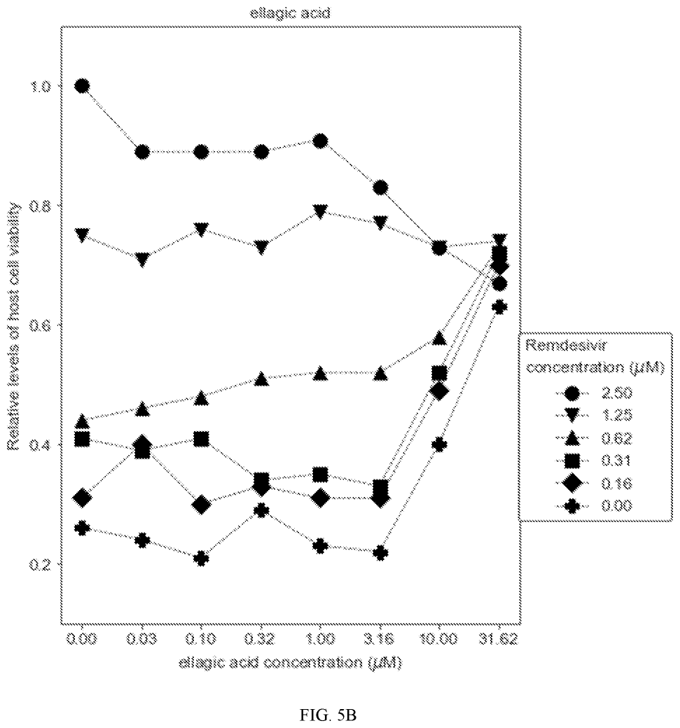

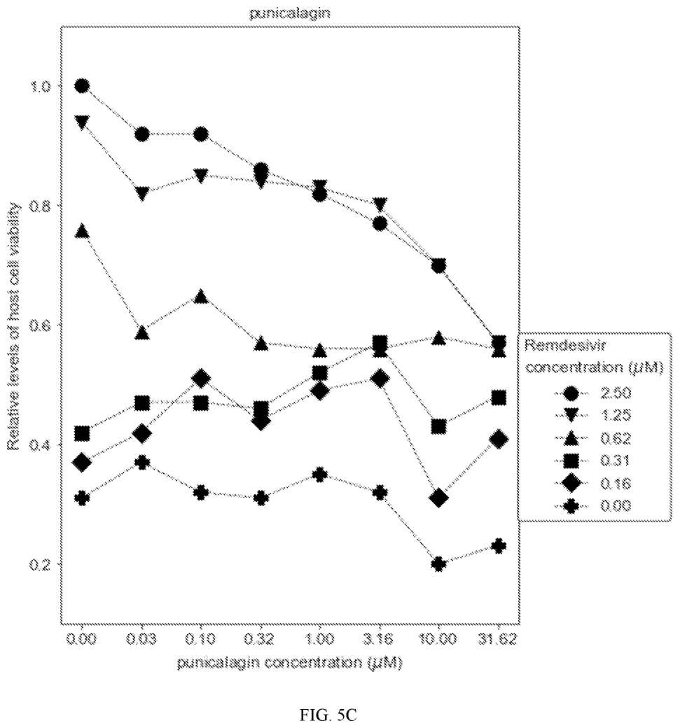

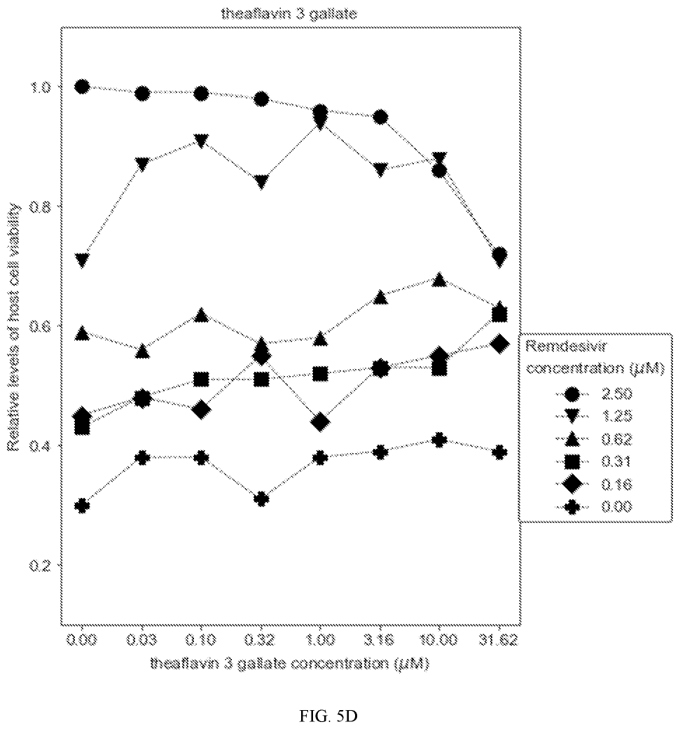

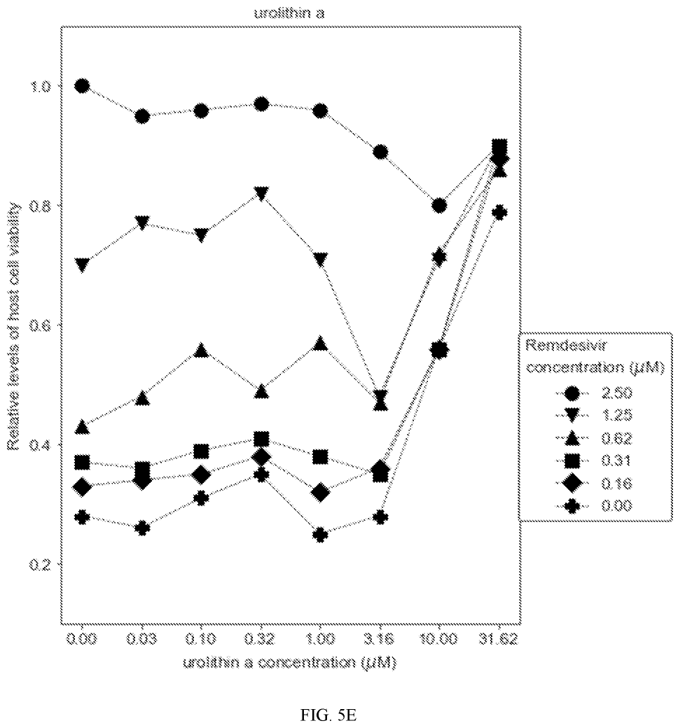

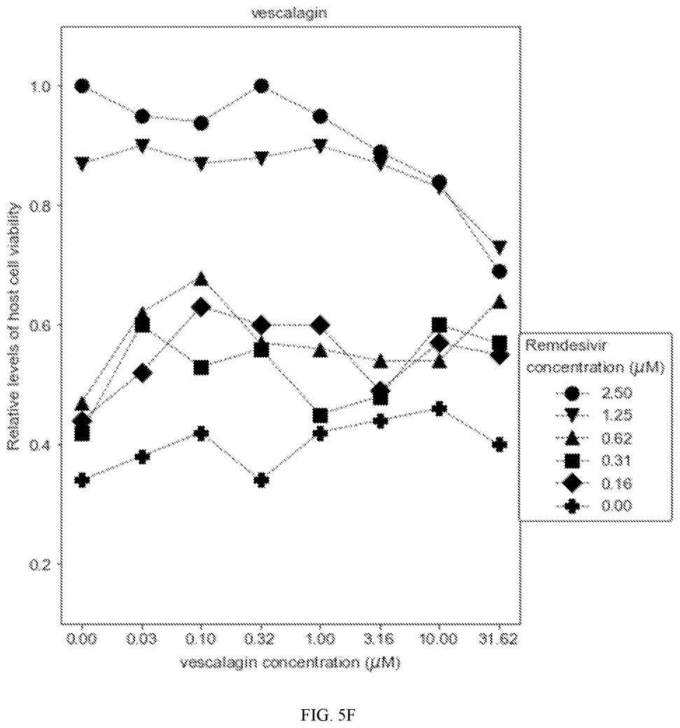

As anticipated, Remdesivir alone improved host cell viability of SARS-CoV infected Vero E6 cells (FIGS. 5A-F). Surprisingly, known metabolites of Punicalagin, Ellagic Acid and Urolithin A, demonstrated an ability to synergize with Remdesivir to further improve host cell viability of SARS-CoV-2-infected Vero E6 cells. Specifically, Ellagic Acid (10 and 31.6 μM) synergized with Remdesivir (at and below 0.625 μM). Urolithin A (10 and 31.6 μM) synergized with Remdesivir (at and below 1.25 μM).

### INCORPORATION BY REFERENCE; EQUIVALENTS

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A method of reducing infection rate of a virus in a subject in need thereof, the method comprising administering to the subject a pharmaceutical composition comprising (a) a compound selected from the group consisting of amentoflavone, bilobetin, ergotamine, ginkgetin, hypericin, punicalagin, theaflavin-3-gallate, urolithin A, urolithin B, ellagic acid, and vescalagin, or a pharmaceutically acceptable salt of any thereof, and (b) a pharmaceutically acceptable carrier or excipient, in a dose and for a time sufficient to reduce the infection rate of the virus in the subject.

2. The method of claim 1, wherein the method reduces coronavirus infection rate in one or more of nasal tissue, bronchi, lung, kidney, esophagus, ileum, colon, rectum, heart, thymus, liver, and blood.

3. The method of claim 1, wherein the method reduces coronavirus infection rate in one or more of epithelial cells, decidual cells, parenchymal cells, and immune cells.

4. The method of any one of claims 1-3, further comprising administering to the subject at least one additional therapeutic agent selected from the group consisting of: a second anti-viral agent, an anti-inflammatory agent, an anticoagulant, and an analgesic.

5. The method of any one of claims 1-3, further comprising administering to the subject at least one additional therapeutic agent selected from Table 2.

6. The method of any one of claims 1-3, wherein the subject has, or is at risk for, a coronavirus infection.

7. The method of claim 6, wherein the coronavirus infection is a Severe Acute Respiratory Syndrome (SARS) infection, a Middle East Respiratory Syndrome (MERS) infection, or a coronavirus 2019 (COVID-19) infection.

8. The method of any one of claims 1-3, where in the virus is SARS-CoV-2.

9. The method of any one of claims 1-3, wherein (a) is amentoflavone.

10. The method of any one of claims 1-3, wherein (a) is bilobetin.

11. The method of any one of claims 1-3, wherein (a) is ergotamine.

12. The method of any one of claims 1-3, wherein (a) is ginkgetin.

13. The method of any one of claims 1-3, wherein (a) is hypericin.

14. The method of any one of claims 1-3, wherein (a) is punicalagin.

15. The method of any one of claims 1-3, wherein (a) is theaflavin-3-gallate.

16. The method of any one of claims 1-3, wherein (a) is urolithin A.

17. The method of claim 16, further comprising administering remdesivir.

18. The method of any one of claims 1-3, wherein (a) is urolithin B.

19. The method of any one of claims 1-3, wherein (a) is ellagic acid.

20. The method of claim 19, further comprising administering remdesivir.

21. The method of any one of claims 1-3, wherein (a) is vescalagin.

22. A method of treating a viral infection in a subject in need thereof, comprising administering to the subject a pharmaceutical composition comprising (a) a compound selected from the group consisting of amentoflavone, bilobetin, ergotamine, ginkgetin, hypericin, punicalagin, theaflavin-3-gallate, urolithin A, urolithin B, ellagic acid, and vescalagin, or a pharmaceutically acceptable salt of any thereof, and (b) a pharmaceutically acceptable carrier or excipient, in a dose and for a time sufficient to treat the viral infection in the subject.

23. The method of claim 22, further comprising administering to the subject at least one additional therapeutic agent selected from the group consisting of: a second anti-viral agent, an anti-inflammatory agent, an anticoagulant, and an analgesic.

24. The method of claim 22, further comprising administering to the subject at least one additional therapeutic agent selected from Table 2.

25. The method of any one of claims 22-24, wherein the viral infection is a coronavirus infection.

26. The method of claim 25, wherein the coronavirus infection is a Severe Acute Respiratory Syndrome (SARS) infection, a Middle East Respiratory Syndrome (MERS) infection, or a coronavirus 2019 (COVID-19) infection.

27. The method of any one of claims 22-24, where in the virus is SARS-CoV-2.

28. The method of any one of claims 22-24, wherein (a) is amentoflavone.

29. The method of any one of claims 22-24, wherein (a) is bilobetin.

30. The method of any one of claims 22-24, wherein (a) is ergotamine.

31. The method of any one of claims 22-24, wherein (a) is ginkgetin.

32. The method of any one of claims 22-24, wherein (a) is hypericin.

33. The method of any one of claims 22-24, wherein (a) is punicalagin.

34. The method of any one of claims 22-24, wherein (a) is theaflavin-3-gallate.

35. The method of any one of claims 22-24, wherein (a) is urolithin A.

36. The method of claim 35, further comprising administering remdesivir.

37. The method of any one of claims 22-24, wherein (a) is urolithin B.

38. The method of any one of claims 22-24, wherein (a) is ellagic acid.

39. The method of claim 38, further comprising administering remdesivir.

40. The method of any one of claims 22-24, wherein (a) is vescalagin.

41. A pharmaceutical composition comprising (a) a compound selected from the group consisting of amentoflavone, bilobetin, ergotamine, ginkgetin, hypericin, punicalagin, theaflavin-3-gallate, urolithin A, urolithin B, ellagic acid, and vescalagin, or a pharmaceutically acceptable salt of any thereof, and (b) a pharmaceutically acceptable carrier or excipient, in a unit dose of between 0.01 mg/kg and 500 mg/kg.

42. The pharmaceutical composition of claim 41, wherein the pharmaceutical composition comprises at least one additional therapeutic agent selected from the group consisting of: a second anti-viral agent, an anti-inflammatory agent, an anticoagulant, and an analgesic.

43. The pharmaceutical composition of claim 41, wherein the pharmaceutical composition comprises at least one additional therapeutic agent selected from Table 2.

44. The pharmaceutical composition of any one of claims 41-43, wherein (a) is amentoflavone.

45. The pharmaceutical composition of any one of claims 41-43, wherein (a) is bilobetin.

46. The pharmaceutical composition of any one of claims 41-43, wherein (a) is ergotamine.

47. The pharmaceutical composition of any one of claims 41-43, wherein (a) is ginkgetin.

48. The pharmaceutical composition of any one of claims 41-43, wherein (a) is hypericin.

49. The pharmaceutical composition of any one of claims 41-43, wherein (a) is punicalagin.

50. The pharmaceutical composition of any one of claims 41-43, wherein (a) is theaflavin-3-gallate.

51. The pharmaceutical composition of any one of claims 41-43, wherein (a) is urolithin A.

52. The pharmaceutical composition of claim 51, further comprising remdesivir.

53. The pharmaceutical composition of any one of claims 41-43, wherein (a) is urolithin B.

54. The pharmaceutical composition of any one of claims 41-43, wherein (a) is ellagic acid.

55. The pharmaceutical composition of claim 54, further comprising remdesivir.

56. The pharmaceutical composition of any one of claims 41-43, wherein (a) is vescalagin.

57. A method of reducing viral infection-induced decrease in cell viability in a subject in need thereof, the method comprising administering to the subject a pharmaceutical composition comprising (a) a compound selected from the group consisting of amentoflavone, bilobetin, ergotamine, ginkgetin, hypericin, punicalagin, theaflavin-3-gallate, urolithin A, urolithin B, ellagic acid, and vescalagin, or a pharmaceutically acceptable salt of any thereof, and (b) a pharmaceutically acceptable carrier or excipient, in a dose and for a time sufficient to reduce the infection rate of the virus in the subject.

58. A method of preventing viral infection-induced cell death in a subject in need thereof, the method comprising administering to the subject a pharmaceutical composition comprising (a) a compound selected from the group consisting of amentoflavone, bilobetin, ergotamine, ginkgetin, hypericin, punicalagin, theaflavin-3-gallate, urolithin A, urolithin B, ellagic acid, and vescalagin, or a pharmaceutically acceptable salt of any thereof, and (b) a pharmaceutically acceptable carrier or excipient, in a dose and for a time sufficient to reduce the infection rate of the virus in the subject.
